(19) United States
(12) Patent Application Publication
(10) Pub. No.: US 2026/0367081 A1
(43) Pub. Date: Nov. 19, 2026

(54) METHODS FOR DIAGNOSING AND TREATING VIRAL AND BACTERIAL SINONASAL INFECTION

(71) Applicant: Diag-Nose Medical Pty Ltd, Notting Hill, Victoria (AU)

(72) Inventors: Shaikh Salah El Din ROSTOM, Glen Waverley, VIC (AU); Adam Michael DAMRY, Ottawa, Ontario (CA); Jonathan LIMPAH, St Kilda, VIC (AU); David Melicio YEN, Bethlehem, PA (US); Brian Shih-Ning WANG, Houston, TX (US); Tanya LUPANCU, North Melbourne, Victoria (AU)

(21) Appl. No.: 19/136,844

(22) PCT Filed: Dec. 8, 2023
(86) PCT No.: PCT/AU2023/051279

§ 371 Date: Jun. 6, 2025

Related U.S. Application Data
(60) Provisional application No. 63/431,124, filed on Dec. 8, 2022.

(51) Int. Cl. G01N 33/569; G01N 33/68

(52) U.S. Cl. CPC G01N 33/56983; G01N 33/6893; G01N 2333/005; G01N 2333/52; G01N 2333/705; G01N 2333/908; G01N 2333/96419; G01N 2800/14; G01N 2800/52; G01N 2800/60; G01N 2800/7095

(57) ABSTRACT

The invention relates to a method for diagnosing a sinonasal inflammatory condition in a patient caused by a viral or bacterial infection. The method comprising measuring concentrations of a combination of biomarkers in a sinonasal sample collected from a patient and comparing the concentration of the biomarkers to one or more reference diagnostic values. The invention also provides a method for establishing reference diagnostic values, method for determining an appropriate treatment, and kits for the diagnosis of sinonasal inflammatory conditions caused by viral and/or bacterial infections.

### TECHNICAL FIELD

The present invention relates to methods and kits for the diagnosis of sinonasal inflammatory conditions caused by viral and bacterial infections. Furthermore, the present invention relates to methods for determining an appropriate treatment for a sinonasal inflammatory condition caused by a viral or bacterial infection and monitoring the response. The methods involve detecting biomarkers in sinonasal samples to assist in the differentiation between viral and bacterial infections and distinguish between disease states.

### BACKGROUND OF THE INVENTION

Sinonasal inflammatory conditions are characterised by the inflammation of mucous membranes that line the nasal cavity (rhinitis), the paranasal sinuses (sinusitis) or both (rhinosinusitis) and are classified according to the duration of symptoms including acute (less than 4 weeks), sub-acute (4-12 weeks) and chronic (over 12 weeks).

Inflammation of the mucous membranes may either lead to nasal drip or may prevent mucous drainage, thereby causing mucous obstruction. Symptoms associated with sinonasal inflammatory conditions include swelling, pressure, and sinus pain. Other symptoms commonly associated with sinonasal inflammatory conditions include sneezing, fever, headaches, loss of smell, sore throat, mucous discharge, and coughing.

Sinonasal inflammatory conditions are prevalent, whereby chronic rhinosinusitis affects around 10% of the population annually. The most common causes of sinonasal inflammatory conditions are viral infections, bacterial infections, and allergic immune responses such as hay-fever. In some rare cases, particularly in people with weakened immune systems, a fungal infection can be the cause.

Despite high prevalence, accurate diagnosis as to the cause of sinonasal inflammatory conditions remains a challenge. Existing modalities to diagnose and determine the cause of sinonasal inflammatory conditions include physical examinations, nasal endoscopies, Computed Tomographic (CT) scans and blood tests.

When a patient presents with symptoms associated with sinonasal inflammatory conditions, a general practitioner (GP) would typically gather patient history and perform a physical examination including anterior rhinoscopy to check for signs such as inflammation or mucosal changes.

Following a physical examination confirming an inflammatory sinonasal condition, tests may be conducted to determine the cause. If an allergen is suspected to be the cause, an allergy test would be conducted. If viral or bacterial infections are suspected as being the cause, sampling techniques including nasopharyngeal swabs may be employed to collect samples from the nasal cavity for subsequent testing. However, typical sampling techniques are insufficient as the nasal cavity is colonised with an abundance of microbiota, therefore potentially resulting in falsely detecting commensal bacteria as pathogenic bacteria.

If the symptoms are severe or persist, the GP would refer the patient to an Ear, Nose and Throat (ENT) specialist. ENT specialists perform nasal endoscopies to examine the sinonasal cavities using specialised tools including endoscopes and speculums. Nasal endoscopy is performed to observe the presence of nasal polyps, swelling, and mucosal changes. A CT scan may be performed in tandem to define the anatomy of the paranasal sinuses and observe any abnormalities.

Additionally, ENT specialists may collect tissue culture samples from the middle meatus (MM), the cavity between the middle turbinate and lateral nasal wall for subsequent testing. The microbiota of the MM has been found to be 80-90% representative of the microbiota in the sinuses and MM samples are less prone to being contaminated by commensal bacteria. However, this modality requires endoscopic vision and is limited to ENT specialists or other trained personnel. Furthermore, this modality is highly invasive, may cause significant discomfort, and may cause bleeding, leading to sample contamination.

Alternative diagnostic methods include collecting blood samples to measure the concentrations of one or more biomarkers associated with viral and bacterial infections. Blood is collected via venepuncture, an invasive process which involves inserting a needle into a superficial vein to draw blood. However, blood is an inadequate proxy for the nasal microenvironment as confounding factors due to comorbidities including cardiovascular disease, trauma, burns or surgical interventions may affect the accurate measurement of biomarkers, therefore affecting the diagnosis. Blood biomarker levels may also be affected by drugs that act systemically. Additionally, the onset of fever can be required for blood biomarker tests which further limits the utility of blood tests.

Accordingly, a need exists for a diagnostic method for sinonasal inflammatory conditions that is non-invasive and does not require site-specific sampling. Furthermore, a need exists for a diagnostic method for sinonasal inflammatory conditions that is accurate, that is not affected by confounding factors and does not require the onset of fever as a symptom.

Any discussion of background art throughout the specification should in no way be considered as an admission that any of the documents or other material referred to was published, known or forms part of the common general knowledge.

Throughout the description and claims of this specification, the word “comprise” and variations of that word, such as “comprising” and “comprises” are not intended to exclude other additives, steps, or integers.

### SUMMARY OF THE INVENTION

In one aspect, the present invention relates to a method for diagnosing a sinonasal inflammatory condition in a patient caused by a viral or bacterial infection comprising measuring concentrations of a combination of biomarkers in a sinonasal sample collected from a patient and comparing the concentration of the biomarkers to a reference diagnostic value, wherein the biomarkers are selected from a panel of biomarkers including Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

In embodiments, the biomarkers are RNA biomarkers. In other embodiments, the one or more biomarkers are protein biomarkers.

In embodiments, the biomarkers for detection of a viral cause of sinonasal inflammation include Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2) and the biomarkers for detection of a bacterial cause of sinonasal inflammation include Human-Defensin 2 (BD-2), C-Reactive Protein (CRP), Eotaxin-2, Eotaxin-3,Granulocyte colony stimulating factor (G-CSF), Interleukin-3 (IL-3), Interleukin-5 (IL-5), Interleukin-8 (IL-8), Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1 (TREM-1), and Triggering receptor expressed on myeloid cells 2 (TREM-2).

In another aspect, the present invention relates to a method for treating a sinonasal inflammatory condition in a patient comprising: determining whether the sinonasal inflammatory condition is caused by a viral infection or a bacterial infection based on measured concentrations of a combination of biomarkers; and administering a therapeutically effective amount of an antibiotic if the patient is identified as having a bacterial infection or administering an antiviral agent if the patient is identified as having a viral infection; wherein the biomarkers are selected from Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

In a further aspect, the present invention relates to a method for monitoring a response of a patient diagnosed with a sinonasal inflammatory condition caused by a bacterial and/or viral infection to a treatment or therapy including: measuring concentrations of a combination of biomarkers in a sinonasal sample following the administration of the treatment or therapy; and comparing the measured concentrations of the biomarkers to reference diagnostic values; wherein the biomarkers are selected from Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

In yet another aspect, the present invention relates to a method for establishing reference diagnostic values to assist with bacterial and/or viral infection caused sinonasal inflammation profiling including: measuring concentrations of at least two biomarkers in sinonasal samples collected from a cohort of patients; wherein the biomarkers are selected from Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2); and wherein the cohort includes at least one patient that is healthy and at least one patient diagnosed with a sinonasal inflammatory condition caused by a viral infection and/or bacterial infection.

Preferably, the cohort includes patients diagnosed with a sinonasal inflammatory condition caused by either a viral infection or a bacterial infection and not both.

In yet another aspect, the present invention relates to a kit for diagnosing sinonasal inflammatory conditions caused by viral or bacterial infections comprising reagents for measuring concentrations of a combination of biomarkers, wherein the biomarkers are selected from Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

In embodiments, the reagents are antibodies. In other embodiments, the reagents are oligonucleotide probes.

In embodiments, the biomarkers for detection of a viral cause of sinonasal inflammation include Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2) and the biomarkers for detection of a bacterial cause of sinonasal inflammation include Human β-Defensin 2 (BD-2), C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-8), Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1(TREM-1), and Triggering receptor expressed on myeloid cells 2 (TREM-2).

In embodiments, the combination of biomarkers includes any one or more of Interleukin-3 (IL-3), Interleukin-5 (IL-5) Matrix metalloproteinase-8 (MMP-8) and Triggering receptor expressed on myeloid cells-1 (TREM-1) and any one or more of Triggering receptor expressed on myeloid cells 2 (TREM-2), Serum amyloid A (SAA), Granzyme B, Interleukin-37 (IL-37), Interferon gamma inducible protein-10 (IP-10), Tumor necrosis factor A (TNFa), and Tumour Necrosis Factor-Related Apoptosis-Inducing Ligand (TRAIL).

In embodiments, the combination of biomarkers includes any one or more of Granulocyte colony stimulating factor (G-CSF) and Myeloperoxidase (MPO), and any one or more of Serum amyloid A (SAA), Granzyme B, Interleukin-37 (IL-37), Interferon gamma inducible protein-10 (IP-10), Tumor necrosis factor A (TNFa), Tumour Necrosis Factor-Related Apoptosis-Inducing Ligand (TRAIL), and Human Myxovirus Resistance Protein 1 (MxA).

In embodiments, the combination of biomarkers includes any one or more of Eotaxin-3 and Matrix metalloproteinase-1 (MMP-1) and any one or more of Serum amyloid A (SAA), Granzyme B, Interferon gamma inducible protein-10 (IP-10), and Tumor necrosis factor A (TNFa).

In embodiments, the combination of biomarkers includes any one or more of Human β-Defensin 2 (BD-2) and Neutrophil Elastase (NE) and any one or more of Human Myxovirus Resistance Protein 1 (MxA), Tumour Necrosis Factor-Related Apoptosis-Inducing Ligand (TRAIL), and Interferon gamma inducible protein-10 (IP-10).

In embodiments, the combination of biomarkers includes Eotaxin-2 and any one or more of Serum amyloid A (SAA), Granzyme B, Interleukin-37 (IL-37), Interferon gamma inducible protein-10 (IP-10), Tumor necrosis factor A (TNFa), and Tumour Necrosis Factor-Related Apoptosis-Inducing Ligand (TRAIL).

In embodiments, the combination of biomarkers includes Human Myxovirus Resistance Protein 1 (MxA) and any one or more of Triggering receptor expressed on myeloid cells 2 (TREM-2) and Serum amyloid A (SAA).

In embodiments, the combination of biomarkers includes Interleukin-8 (IL-8) and any one or more of Triggering receptor expressed on myeloid cells 2 (TREM-2), Serum amyloid A (SAA), Granzyme B, Interleukin-37 (IL-37), Interferon gamma inducible protein-10 (IP-10), Tumor necrosis factor A (TNFa), Tumour Necrosis Factor-Related Apoptosis-Inducing Ligand (TRAIL), and Human Myxovirus Resistance Protein 1 (MxA).

In embodiments, the combination of biomarkers includes C-reactive protein (CRP) and any one or more of Interferon gamma inducible protein-10 (IP-10) and Human Myxovirus Resistance Protein 1 (MxA).

In embodiments, the combination of biomarkers is Interleukin-10 and Tumour Necrosis Factor-Related Apoptosis-Inducing Ligand (TRAIL).

In embodiments, the sinonasal sample is sinonasal secretions, sinonasal aspirates or tissue cultures.

In embodiments, the one or more biomarkers are RNA biomarkers.

In embodiments, the one or more biomarkers are protein biomarkers.

In embodiments, the present invention includes kits for diagnosing sinonasal inflammatory conditions caused by viral or bacterial infections adapted for use with any one or more of multiplexing instruments, antibody/aptamer binding immunoassays, point-of-care microfluidic devices and lateral flow assays.

### BRIEF DESCRIPTION OF THE FIGURES

The present invention will now be described in more detail with reference to preferred embodiments illustrated in the accompanying figures, wherein:

FIG. 1 illustrates the distribution of BD-2 protein concentrations measured across patient groups in Study 1 according to the experimental method described herein below. Positive corresponds to bacterial patient group, Challenge corresponds to viral patient group, and Negative corresponds to Healthy patient group.

FIG. 2 illustrates an ROC curve for BD-2 derived from the data of FIG. 1.

FIG. 3 illustrates the distribution of CRP protein concentrations measured across patient groups in Study 1 according to the experimental method described herein below. Positive corresponds to bacterial patient group, Challenge corresponds to viral patient group, and Negative corresponds to Healthy patient group.

FIG. 4 illustrates an ROC curve for CRP derived from the data of FIG. 3.

FIG. 5 illustrates the distribution of G-CSF protein concentrations measured across patient groups in Study 1 according to the experimental method described herein below. Positive corresponds to bacterial patient group, Challenge corresponds to viral patient group, and Negative corresponds to Healthy patient group.

FIG. 6 illustrates an ROC curve for G-CSF derived from the data of FIG. 5.

FIG. 7 illustrates the distribution of IL-8 protein concentrations measured across patient groups in Study 1 according to the experimental method described herein below. Positive corresponds to bacterial patient group, Challenge corresponds to viral patient group, and Negative corresponds to Healthy patient group.

FIG. 8 illustrates an ROC curve for IL-8 derived from the data of FIG. 7.

FIG. 9 illustrates the distribution of MPO protein concentrations measured across patient groups in Study 1 according to the experimental method described herein below. Positive corresponds to bacterial patient group, Challenge corresponds to viral patient group, and Negative corresponds to Healthy patient group.

FIG. 10 illustrates an roc curve for mpo derived from the data of FIG. 9.

FIG. 11 illustrates the distribution of NE protein concentrations measured across patient groups in Study 1 according to the experimental method described herein below. Positive corresponds to bacterial patient group, Challenge corresponds to viral patient group, and Negative corresponds to Healthy patient group.

FIG. 12 illustrates an ROC curve for NE derived from the data of FIG. 11.

FIG. 13 illustrates the distribution of SAA protein concentrations measured across patient groups in Study 1 according to the experimental method described herein below. Positive corresponds to bacterial patient group, Challenge corresponds to viral patient group, and Negative corresponds to Healthy patient group.

FIG. 14 illustrates an ROC curve for SAA derived from the data of FIG. 13.

FIG. 15 illustrates the distribution of TREM-2 protein concentrations measured across patient groups in Study 1 according to the experimental method described herein below. Positive corresponds to bacterial patient group, Challenge corresponds to viral patient group, and Negative corresponds to Healthy patient group.

FIG. 16 illustrates an ROC curve for TREM-2 derived from the data of FIG. 15.

FIG. 17 illustrates the distribution of IP-10 protein concentrations measured across patient groups in Study 1 according to the experimental method described herein below. Positive corresponds to viral patient group, Challenge corresponds to bacterial patient group, and Negative corresponds to Healthy patient group.

FIG. 18 illustrates an ROC curve for IP-10 derived from the data of FIG. 17.

FIG. 19 illustrates the distribution of MxA protein concentrations measured across patient groups in Study 1 according to the experimental method described herein below. Positive corresponds to viral patient group, Challenge corresponds to bacterial patient group, and Negative corresponds to Healthy patient group.

FIG. 20 illustrates an roc curve for MxA obtained from the data of FIG. 19.

FIG. 21 illustrates the distribution of TRAIL protein concentrations measured across patient groups in Study 1 according to the experimental method described herein below. Positive corresponds to viral patient group, Challenge corresponds to bacterial patient group, and Negative corresponds to Healthy patient group.

FIG. 22 illustrates an ROC curve for TRAIL obtained from the data of FIG. 21.

FIG. 23 comprises a table, identified as Table 1, which includes cut off values and associated sensitivity and specificity values for diagnosing a bacterial infection or viral infection in Study 1 using the bacterial biomarkers Human β-Defensin 2 (BD-2), C-Reactive Protein (CRP), Granulocyte colony stimulating factor (G-CSF), Interleukin-8 (IL-8), Myeloperoxidase (MPO), Neutrophil Elastase (NE), Serum amyloid A (SAA), and Triggering receptor expressed on myeloid cells 2 (TREM-2), and viral biomarkers Interferon gamma inducible protein-10 (IP-10), Human Myxovirus Resistance Protein 1 (MxA), and Tumour Necrosis Factor-Related Apoptosis-Inducing Ligand (TRAIL).

FIG. 24 comprises a table, identified as Table 2, which includes cut off values and associated sensitivity and specificity data for diagnosing a bacterial infection or viral infection in Study 1 using combinations of biomarkers selected from Human Myxovirus Resistance Protein 1 (MxA), Neutrophil Elastase (NE), Interferon gamma inducible protein-10 (IP-10), Human β-Defensin 2 (BD-2), Interleukin-8 (IL-8), Myeloperoxidase (MPO), C-Reactive Protein (CRP), Tumour Necrosis Factor-Related Apoptosis-Inducing Ligand (TRAIL), Triggering receptor expressed on myeloid cells 2 (TREM-2); Granulocyte colony stimulating factor (G-CSF) and Serum amyloid A (SAA).

FIG. 25 illustrates the distribution of CRP protein concentrations measured across patient groups in Study 2 according to the experimental method described herein below. Positive corresponds to bacterial patient group, Challenge corresponds to viral patient group, and Negative corresponds to Healthy patient group.

FIG. 26 illustrates an ROC curve for CRP derived from the data of FIG. 25.

FIG. 27 illustrates the distribution of Eotaxin-2 protein concentrations measured across patient groups in Study 2 according to the experimental method described herein below. Positive corresponds to bacterial patient group, Challenge corresponds to viral patient group, and Negative corresponds to Healthy patient group.

FIG. 28 illustrates an ROC curve for Eotaxin-2 derived from the data of FIG. 27.

FIG. 29 illustrates the distribution of Eotaxin-3 protein concentrations measured across patient groups in Study 2 according to the experimental method described herein below. Positive corresponds to bacterial patient group, Challenge corresponds to viral patient group, and Negative corresponds to Healthy patient group.

FIG. 30 illustrates an ROC curve for Eotaxin-3 derived from the data of FIG. 29.

FIG. 31 illustrates the distribution of G-CSF protein concentrations measured across patient groups in Study 2 according to the experimental method described herein below. Positive corresponds to bacterial patient group, Challenge corresponds to viral patient group, and Negative corresponds to Healthy patient group.

FIG. 32 illustrates an ROC curve for G-CSF derived from the data of FIG. 31.

FIG. 33 illustrates the distribution of IL-3 protein concentrations measured across patient groups in Study 2 according to the experimental method described herein below. Positive corresponds to bacterial patient group, Challenge corresponds to viral patient group, and Negative corresponds to Healthy patient group.

FIG. 34 illustrates an ROC curve for IL-3 derived from the data of FIG. 33.

FIG. 35 illustrates the distribution of IL-5 protein concentrations measured across patient groups in Study 2 according to the experimental method described herein below. Positive corresponds to bacterial patient group, Challenge corresponds to viral patient group, and Negative corresponds to Healthy patient group.

FIG. 36 illustrates an ROC curve for IL-5 derived from the data of FIG. 35.

FIG. 37 illustrates the distribution of IL-8 protein concentrations measured across patient groups in Study 2 according to the experimental method described herein below. Positive corresponds to bacterial patient group, Challenge corresponds to viral patient group, and Negative corresponds to Healthy patient group.

FIG. 38 illustrates an ROC curve for IL-8 derived from the data of FIG. 37.

FIG. 39 illustrates the distribution of IL-10 protein concentrations measured across patient groups in Study 2 according to the experimental method described herein below. Positive corresponds to bacterial patient group, Challenge corresponds to viral patient group, and Negative corresponds to Healthy patient group.

FIG. 40 illustrates an ROC curve for IL-10 derived from the data of FIG. 39.

FIG. 41 illustrates the distribution of MMP-1 protein concentrations measured across patient groups in Study 2 according to the experimental method described herein below. Positive corresponds to bacterial patient group, Challenge corresponds to viral patient group, and Negative corresponds to Healthy patient group.

FIG. 42 illustrates an ROC curve for MMP-1 derived from the data of FIG. 41.

FIG. 43 illustrates the distribution of MMP-8 protein concentrations measured across patient groups in Study 2 according to the experimental method described herein below. Positive corresponds to bacterial patient group, Challenge corresponds to viral patient group, and Negative corresponds to Healthy patient group.

FIG. 44 illustrates an ROC curve for MMP-8 derived from the data of FIG. 43.

FIG. 45 illustrates the distribution of MPO protein concentrations measured across patient groups in Study 2 according to the experimental method described herein below. Positive corresponds to bacterial patient group, Challenge corresponds to viral patient group, and Negative corresponds to Healthy patient group.

FIG. 46 illustrates an ROC curve for MPO derived from the data of FIG. 45.

FIG. 47 illustrates the distribution of TREM-1 protein concentrations measured across patient groups in Study 2 according to the experimental method described herein below. Positive corresponds to bacterial patient group, Challenge corresponds to viral patient group, and Negative corresponds to Healthy patient group.

FIG. 48 illustrates an ROC curve for TREM-1 derived from the data of FIG. 47.

FIG. 49 illustrates the distribution of Granzyme B protein concentrations measured across patient groups in Study 2 according to the experimental method described herein below. Positive corresponds to viral patient group, Challenge corresponds to bacterial patient group, and Negative corresponds to Healthy patient group.

FIG. 50 illustrates an ROC curve for Granzyme B derived from the data of FIG. 49.

FIG. 51 illustrates the distribution of IL-37 protein concentrations measured across patient groups in Study 2 according to the experimental method described herein below. Positive corresponds to viral patient group, Challenge corresponds to bacterial patient group, and Negative corresponds to Healthy patient group.

FIG. 52 illustrates an ROC curve for IL-37 derived from the data of FIG. 50.

FIG. 53 illustrates the distribution of IP-10 protein concentrations measured across patient groups in Study 2 according to the experimental method described herein below. Positive corresponds to viral patient group, Challenge corresponds to bacterial patient group, and Negative corresponds to Healthy patient group.

FIG. 54 illustrates an ROC curve for IP-10 derived from the data of FIG. 53.

FIG. 55 illustrates the distribution of SAA protein concentrations measured across patient groups in Study 2 according to the experimental method described herein below. Positive corresponds to viral patient group, Challenge corresponds to bacterial patient group, and Negative corresponds to Healthy patient group.

FIG. 56 illustrates an ROC curve for SAA derived from the data of FIG. 55.

FIG. 57 illustrates the distribution of TNFa protein concentrations measured across patient groups in Study 2 according to the experimental method described herein below. Positive corresponds to viral patient group, Challenge corresponds to bacterial patient group, and Negative corresponds to Healthy patient group.

FIG. 58 illustrates an ROC curve for TNFa derived from the data of FIG. 57.

FIG. 59 illustrates the distribution of TRAIL protein concentrations measured across patient groups in Study 2 according to the experimental method described herein below. Positive corresponds to viral patient group, Challenge corresponds to bacterial patient group, and Negative corresponds to Healthy patient group.

FIG. 60 illustrates an ROC curve for TRAIL derived from the data of FIG. 59.

FIG. 61 illustrates the distribution of Granzyme B protein concentrations measured across patient groups in Study 2 according to the experimental method described herein below. Positive corresponds to viral patient group, Challenge corresponds to bacterial patient group, and Negative corresponds to Healthy patient group.

FIG. 62 illustrates an ROC curve for Granzyme B derived from the data of FIG. 61.

FIG. 63 comprises a table, identified as Table 3, which includes cut off values and associated sensitivity and specificity values for diagnosing a bacterial infection or viral infection in Study 2 using the bacterial biomarkers Human 1-C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-8), Interleukin-10 (IL-10), Matrix metalloproteinase-1 (MMP-1), Matrix metalloproteinase-8 (MMP-8), Myeloperoxidase (MPO), and Triggering receptor expressed on myeloid cells 1 (TREM-1), and viral biomarkers Granzyme B, Interleukin-37 (IL-37), Interferon gamma inducible protein-10 (IP-10), Serum Amyloid A (SAA), Tumor necrosis factor A (TNFa), Tumor necrosis factor-related apoptosis-inducing ligand (TRAIL), and Tumour Necrosis Factor-Related Apoptosis-Inducing Ligand (TRAIL).

FIG. 64 comprises a table, identified as Table 4, which includes cut off values and associated sensitivity and specificity data for diagnosing a bacterial infection or viral infection in Study 2 using combinations of biomarkers selected from C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2)

FIG. 65 illustrates the distribution of BD-2 protein concentrations measured across patient groups in Study 3 according to the experimental method described herein below. Positive corresponds to bacterial patient group, Challenge corresponds to viral patient group, and Negative corresponds to Healthy patient group.

FIG. 66 illustrates an ROC curve for BD-2 derived from the data of FIG. 65.

FIG. 67 illustrates the distribution of CRP protein concentrations measured across patient groups in Study 3 according to the experimental method described herein below. Positive corresponds to bacterial patient group, Challenge corresponds to viral patient group, and Negative corresponds to Healthy patient group.

FIG. 68 illustrates an ROC curve for CRP derived from the data of FIG. 67.

FIG. 69 illustrates the distribution of G-CSF protein concentrations measured across patient groups in Study 3 according to the experimental method described herein below. Positive corresponds to bacterial patient group, Challenge corresponds to viral patient group, and Negative corresponds to Healthy patient group.

FIG. 70 illustrates an ROC curve for G-CSF derived from the data of FIG. 69.

FIG. 71 illustrates the distribution of IL-8 protein concentrations measured across patient groups in Study 3 according to the experimental method described herein below. Positive corresponds to bacterial patient group, Challenge corresponds to viral patient group, and Negative corresponds to Healthy patient group.

FIG. 72 illustrates an ROC curve for IL-8 derived from the data of FIG. 71.

FIG. 73 illustrates the distribution of MPO protein concentrations measured across patient groups in Study 3 according to the experimental method described herein below. Positive corresponds to bacterial patient group, Challenge corresponds to viral patient group, and Negative corresponds to Healthy patient group.

FIG. 74 illustrates an ROC curve for MPO derived from the data of FIG. 73.

FIG. 75 illustrates the distribution of NE protein concentrations measured across patient groups in Study 3 according to the experimental method described herein below. Positive corresponds to bacterial patient group, Challenge corresponds to viral patient group, and Negative corresponds to Healthy patient group.

FIG. 76 illustrates an ROC curve for NE derived from the data of FIG. 75.

FIG. 77 illustrates the distribution of SAA protein concentrations measured across patient groups in Study 3 according to the experimental method described herein below. Positive corresponds to bacterial patient group, Challenge corresponds to viral patient group, and Negative corresponds to Healthy patient group.

FIG. 78 illustrates an ROC curve for SAA derived from the data of FIG. 77.

FIG. 79 illustrates the distribution of TREM-2 protein concentrations measured across patient groups in Study 3 according to the experimental method described herein below. Positive corresponds to bacterial patient group, Challenge corresponds to viral patient group, and Negative corresponds to Healthy patient group.

FIG. 80 illustrates an ROC curve for TREM-2 derived from the data of FIG. 79.

FIG. 81 illustrates the distribution of IP-10 protein concentrations measured across patient groups in Study 3 according to the experimental method described herein below. Positive corresponds to viral patient group, Challenge corresponds to bacterial patient group, and Negative corresponds to Healthy patient group.

FIG. 82 illustrates an ROC curve for IP-10 derived from the data of FIG. 81.

FIG. 83 illustrates the distribution of MxA protein concentrations measured across patient groups in Study 3 according to the experimental method described herein below. Positive corresponds to viral patient group, Challenge corresponds to bacterial patient group, and Negative corresponds to Healthy patient group.

FIG. 84 illustrates an ROC curve for MxA obtained from the data of FIG. 83.

FIG. 85 illustrates the distribution of TRAIL protein concentrations measured across patient groups in Study 3 according to the experimental method described herein below. Positive corresponds to viral patient group, Challenge corresponds to bacterial patient group, and Negative corresponds to Healthy patient group.

FIG. 86 illustrates an ROC curve for TRAIL obtained from the data of FIG. 85.

FIG. 87 comprises a table, identified as Table 5, which includes cut off values and associated sensitivity and specificity values for diagnosing a bacterial infection or viral infection in Study 3 using the bacterial biomarkers Human β-Defensin 2 (BD-2), C-Reactive Protein (CRP), Granulocyte colony stimulating factor (G-CSF), Interleukin-8 (IL-8), Myeloperoxidase (MPO), Neutrophil Elastase (NE), Serum amyloid A (SAA), and Triggering receptor expressed on myeloid cells 2 (TREM-2), and viral biomarkers Interferon gamma inducible protein-10 (IP-10), Human Myxovirus Resistance Protein 1 (MxA), and Tumour Necrosis Factor-Related Apoptosis-Inducing Ligand (TRAIL).

FIG. 88 comprises a table, identified as Table 6, which includes cut off values and associated sensitivity and specificity data for diagnosing a bacterial infection or viral infection in Study 3 using combinations of biomarkers selected from Human Myxovirus Resistance Protein 1 (MxA), Neutrophil Elastase (NE), Interferon gamma inducible protein-10 (IP-10), Human β-Defensin 2 (BD-2), Interleukin-8 (IL-8), Myeloperoxidase (MPO), C-Reactive Protein (CRP), Tumour Necrosis Factor-Related Apoptosis-Inducing Ligand (TRAIL), Triggering receptor expressed on myeloid cells 2 (TREM-2); Granulocyte colony stimulating factor (G-CSF) and Serum amyloid A (SAA).

### DETAILED DESCRIPTION

### Definitions

Unless otherwise defined, all technical and scientific terms used herein have the same meaning as understood in the art.

The term “biomarker” as used herein refers to a biological characteristic that is a quantitative indicator of biological processes, pathological processes, and pharmacological responses. Biomarkers are measured in biological samples such as bodily fluids and encompass genes, proteins, nucleic acids, fragments of nucleic acids, lipids, metabolites, carbohydrates, and hormones.

Biomarkers referred to herein include Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

The terms “patient” and “subject” are used herein interchangeably unless specified otherwise and refer to any animal or cells thereof including human and non-human animals. In preferred embodiments, the patient or subject is human.

### Embodiments of the Invention

The present invention relates to methods and kits for the diagnosis and treatment of sinonasal inflammatory conditions caused by viral and bacterial infections. The methods include diagnosing sinonasal inflammatory conditions caused by viral and bacterial infections; determining an appropriate treatment for a sinonasal inflammatory condition caused by a viral or bacterial infection; and monitoring a patient's response to treatment.

Each of the methods involve a) collecting sinonasal samples from a patient; b) detecting and measuring the concentration of biomarkers in the sinonasal sample associated with viral infections and bacterial infections; and c) comparing the measured concentrations to reference diagnostic values.

The present invention further relates to a method of establishing reference diagnostic values to assist with sinonasal inflammatory profiling.

### Samples

The methods disclosed herein describe combinations of biomarkers detected in sinonasal samples for the accurate diagnosis of sinonasal inflammatory conditions that do not require invasive or site-specific sampling.

### Diagnosis

In one aspect, the present invention relates to a method for the accurate diagnosis of sinonasal inflammatory conditions caused by viral and bacterial infections. The method comprises a) collecting sinonasal samples from a patient; b) detecting and measuring the concentration of biomarkers in the biological sample that are associated with viral infections and bacterial infections; and c) analysing and comparing the measured concentrations of the biomarkers to reference diagnostic values.

Sinonasal samples include sinonasal secretions, sinonasal aspirates and sinonasal tissue cultures. Preferably, the sinonasal sample is collected by forced exhalation or absorptive strips. In embodiments, the dilution ratio of the sinonasal sample is a standardised dilution ratio for accurate testing.

### Biomarkers

In embodiments, the invention disclosed herein includes application of a sinonasal sample for the detection of one or more biomarkers that are upregulated in a patient exhibiting sinonasal inflammation caused by a viral or a bacterial infection.

In preferred embodiments, the biomarkers detected and measured are selected from a protein biomarker panel comprising Human-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2). At least 2 biomarkers are selected from the biomarker panel. In other embodiments, the biomarkers are RNA.

Biomarkers for detection of a viral cause of sinonasal inflammation include Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2).

Biomarkers for detection of a bacterial cause of sinonasal inflammation include Human-Defensin 2 (BD-2), C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-8), Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1 (TREM-1), and Triggering receptor expressed on myeloid cells 2 (TREM-2).

### Assays

In embodiments, the biomarkers are detected and measured using multiplexing kits such as LUMINEX multiplex, Meso Scale Discovery (MSD), Quanterix and Olink. In other embodiments, the biomarkers are detected and measured using ELISA, Western Blot, or flow cytometry. In further embodiments, point-of-care systems such as microfluidic chips or multiplexing lateral flow test strips are used to the detect and measure the biomarkers in vitro. However, it is to be appreciated that other techniques can be used to detect and measure biomarker concentrations.

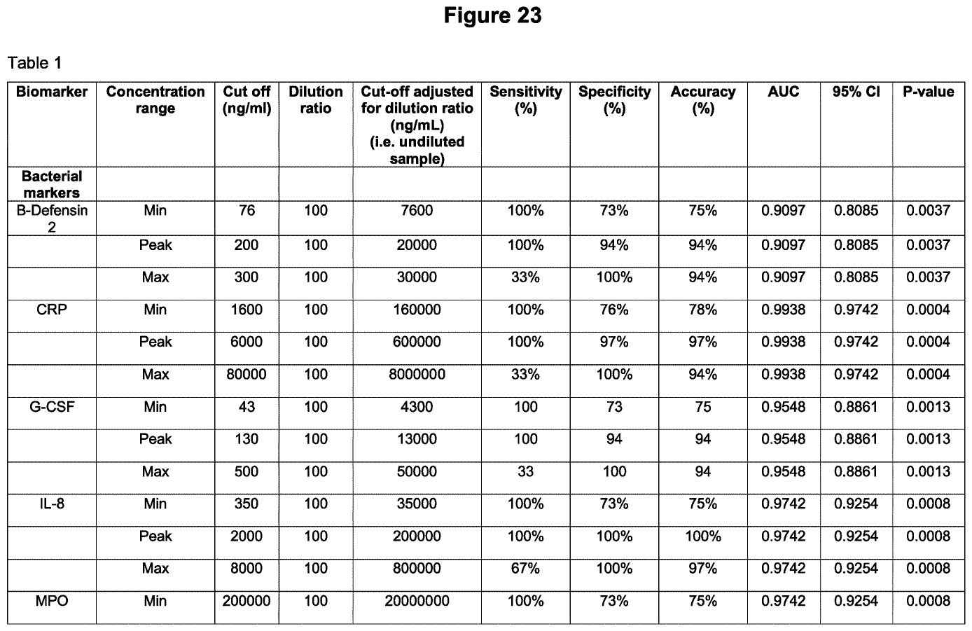

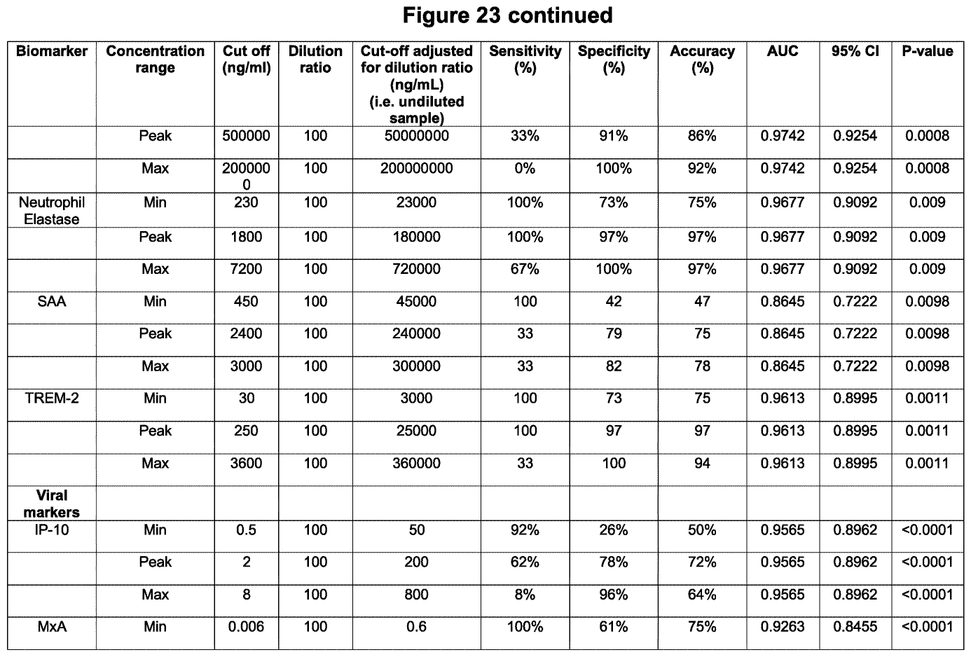

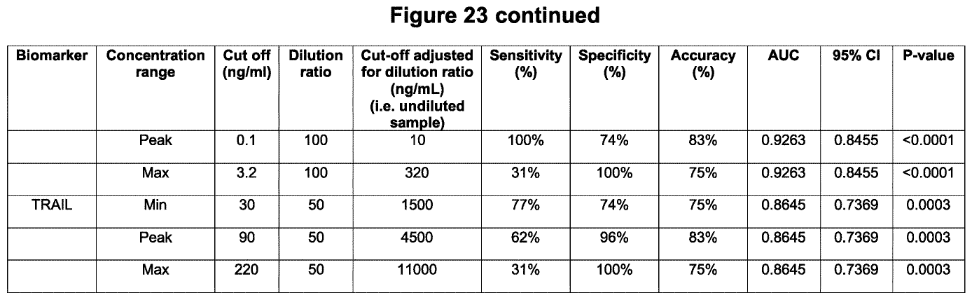

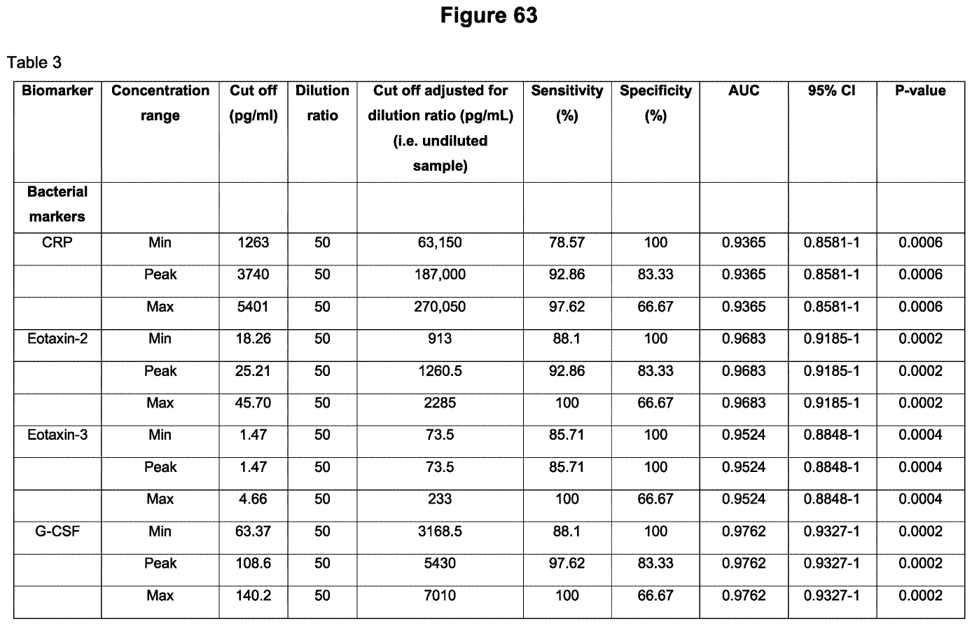

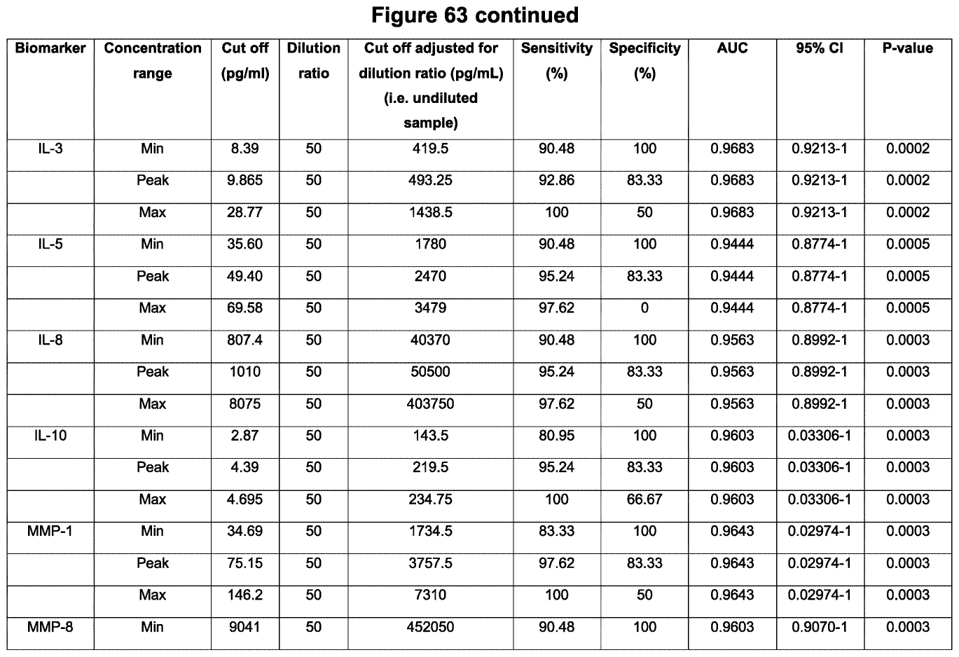

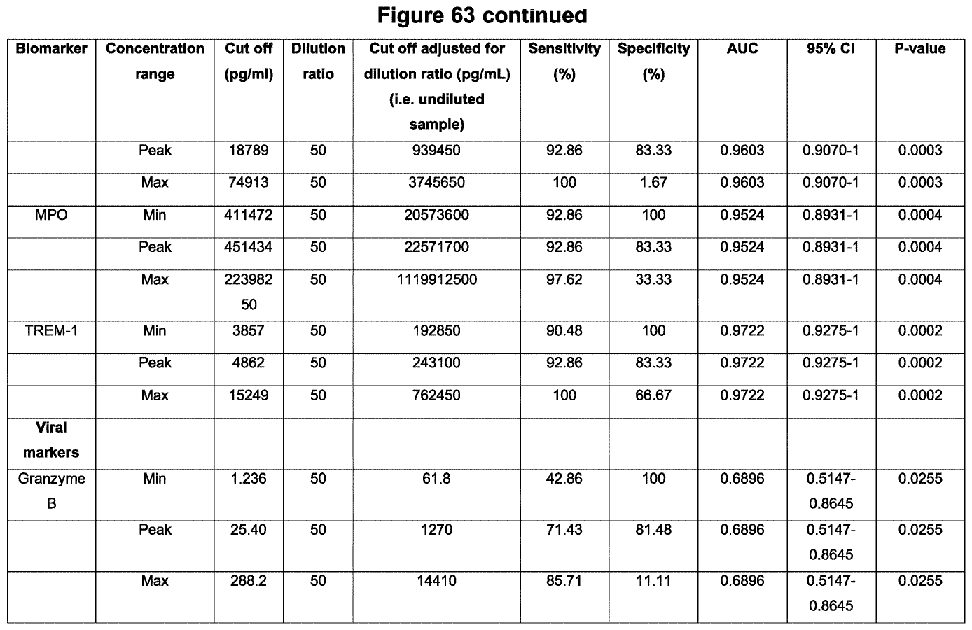

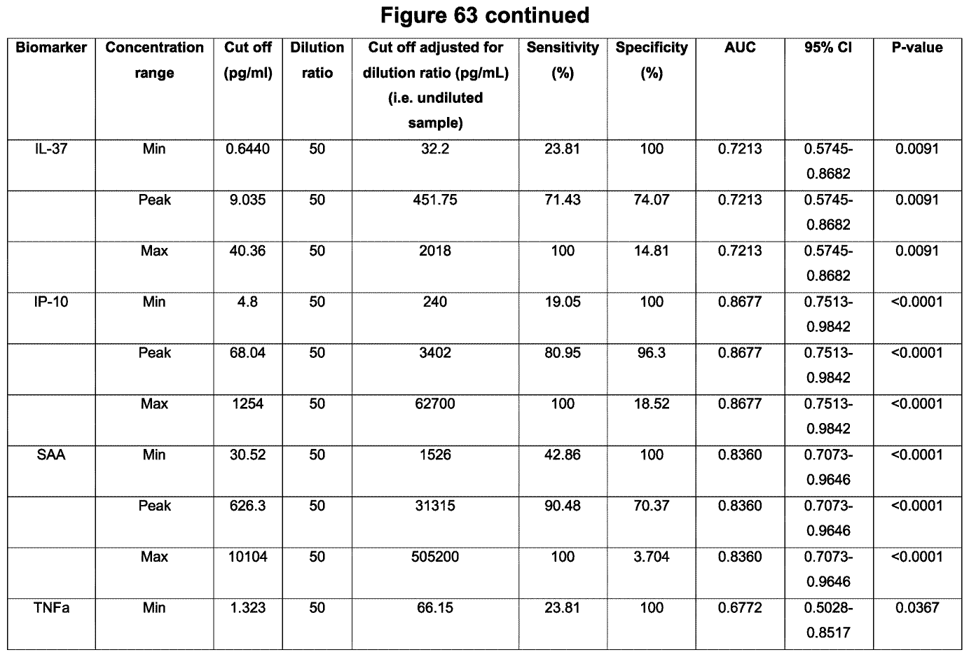

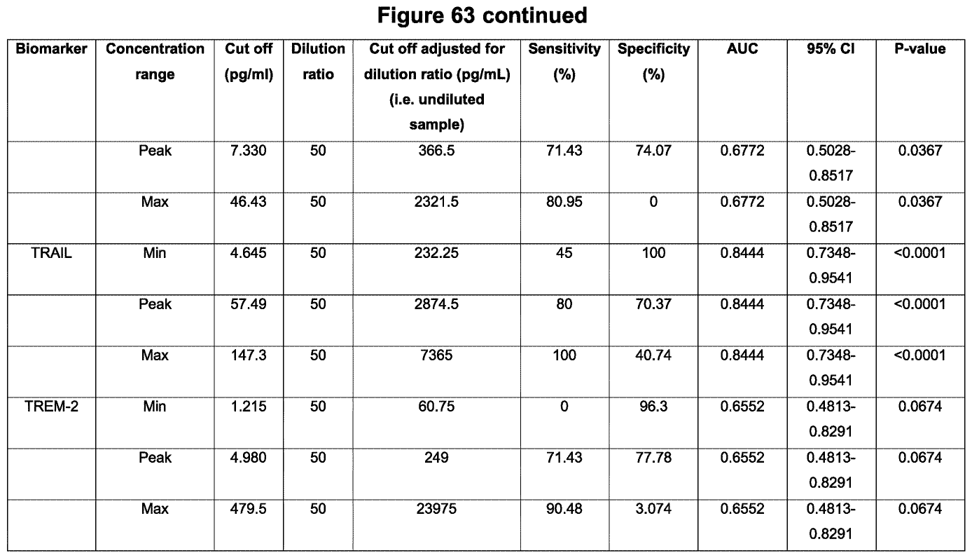

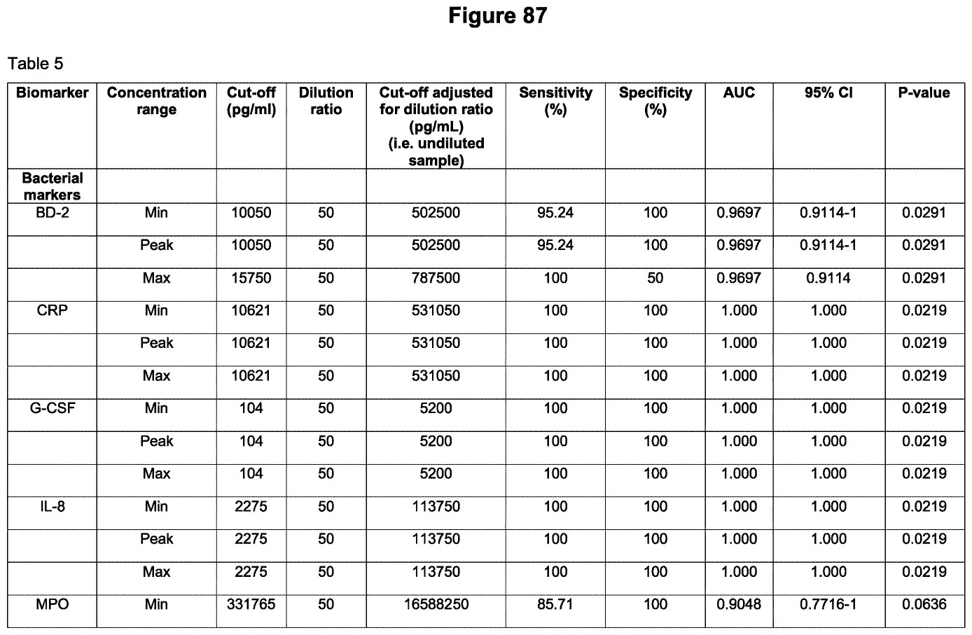

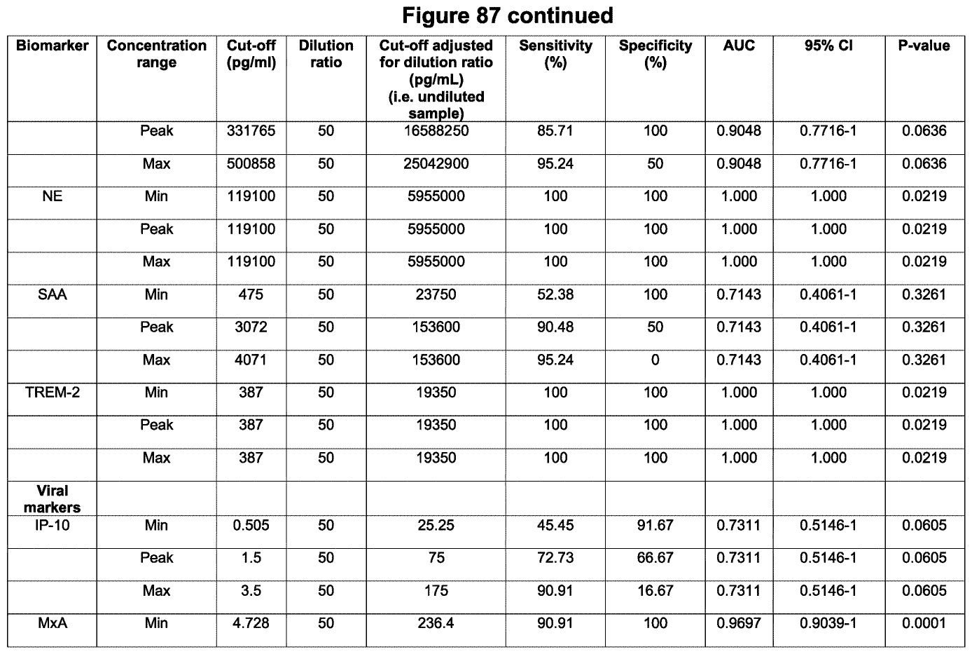

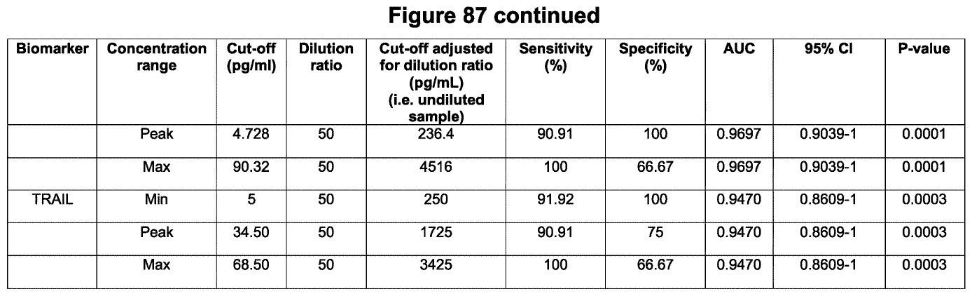

FIG. 23, FIG. 63, and FIG. 87 contains Table 1, Table 3, and Table 5, respectively. These tables include cut off values and associated sensitivity and specificity values of biomarkers for bacterial infection or viral infection obtained from data derived from samples obtained from different studies according to the experimental method described herein below (FIG. 23 from Study 1, FIG. 62 from Study 2, and FIG. 87 from Study 3). The tables illustrate the cut-off concentrations of biomarkers in samples diluted at a specified dilution ratio or undiluted samples, wherein at Min, Peak, and Max cutoff concentrations, the Min Peak, and Max specificity and sensitivity are specified. The tables also set out additional data for each of the biomarkers, including accuracy, area under the curve (AUC), 95% confidence interval, and P-value.

For example and with reference to FIG. 23 (Table 1), the cut-off concentrations of CRP in an undiluted sample, wherein at a minimum cut-off concentration of 160,000, specificity is 76% and sensitivity is 100% for bacterial infection, and wherein at a maximum cut-off concentration of 8,000,000 ng/ml, specificity is 100% and sensitivity is 33% for bacterial infection, and wherein at a peak cut-off concentration of 600,000 ng/ml, specificity is 97% and sensitivity is 100% for bacterial infection. That is, 1,600 to 80,000 ng/ml with a peak of 6,000 ng/ml in a sample with a dilution ratio of 1:100.

The cutoff concentrations in FIGS. 23, 63, and 87 also represent the reference diagnostic range thresholds identified in the different studies. For example and with reference to FIG. 23 (Table 1), in undiluted samples in Study 1, a concentration of β-Defensin 2 within a range of 7,600 to 30,000 ng/ml is indicative of bacterial infection. A concentration of CRP within a range of 160,000 ng/ml to 8,000,000 ng/ml is indicative of bacterial infection. A concentration of G-CSF within a range of 4,300 to 50,000 ng/ml is indicative of bacterial infection. A concentration of IL-8 within a range of 35,000 to 800,000 ng/ml is indicative of bacterial infection. A concentration of MPO within a range of 20,000,000 to 200,000,000 ng/ml is indicative of bacterial infection. A concentration of neutrophil elastase within a range of 23,000 to 720,000 ng/ml is indicative of bacterial infection. A concentration of SAA within a range of 45,000 to 300,000 ng/ml is indicative of bacterial infection. A concentration of TREM-2 within a range of 3,000 to 360,000 ng/ml is indicative of bacterial infection.

Furthermore, with reference to FIG. 23 (Table 1), a concentration of IP-10 within a range of 50 to 800 ng/ml is indicative of viral infection. A concentration of MxA within a range of 0.6 to 320 ng/ml is indicative of viral infection. A concentration of TRAIL within a range of 3,000 to 22,000 ng/ml is indicative of viral infection.

### Treating

In another aspect, the present invention relates to determining an appropriate treatment for a patient diagnosed with a sinonasal inflammatory condition caused by a viral or bacterial infection. The method comprises: a) collecting a biological sample from a patient; b) detecting and measuring the concentration of biomarkers in the biological sample that are associated with viral infections and bacterial infections; c) analysing and comparing the measured concentrations of the biomarkers to reference diagnostic values; and d) administering a therapeutically effective amount of an antibiotic if the patient is diagnosed with a sinonasal inflammatory condition caused by a bacterial infection or administering a therapeutically effective amount of an antiviral if the patient is diagnosed with a sinonasal inflammatory condition caused by a virus.

Sinonasal samples include sinonasal secretions, sinonasal aspirates and sinonasal tissue cultures. Preferably, the sinonasal sample is collected by forced exhalation or absorptive strips. In embodiments, the volume of the sinonasal sample is a standardised sample volume consistent with volumes required for accurate testing.

In preferred embodiments. the biomarkers detected and measured are selected from a protein biomarker panel comprising Human-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2). At least 2 biomarkers are selected from the biomarker panel.

In embodiments, the biomarkers are detected and measured using multiplexing kits such as LUMINEX multiplex, Meso Scale Discovery (MSD), Quanterix and Olink. In other embodiments, the biomarkers are detected and measured using ELISA, Western Blot, or flow cytometry. In further embodiments, point-of-care systems such as microfluidic chips or multiplexing lateral flow test strips are used to detect and measure the biomarkers. However, it is to be appreciated that other techniques can be used to detect and measure biomarker concentrations.

### Monitoring

In yet another aspect, the invention relates to a method for monitoring the response of a patient diagnosed with a sinonasal inflammatory condition caused by a viral or bacterial infection and administered a therapy or treatment. The method comprises: a) collecting a biological sample from a patient; b) measuring the concentration of biomarkers in the biological sample that are associated with viral infections and/or bacterial infections; and c) analysing and comparing the measured concentrations of the biomarkers to reference diagnostic values.

Sinonasal samples include sinonasal secretions, sinonasal aspirates and sinonasal tissue cultures. Preferably, the sinonasal sample is collected by forced exhalation or absorptive strips. In embodiments, the volume of the sinonasal sample is a standardised sample volume consistent with volumes required for accurate testing.

In preferred embodiments, the biomarkers detected and measured are selected from a protein biomarker panel comprising Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2). At least 2 biomarkers are selected from the biomarker panel. In other embodiments, the biomarkers are RNA.

In embodiments, the biomarkers are detected and measured using multiplexing instruments comprising bead or planar arrays such as LUMINEX and Quanterix instruments, electrochemiluminescence such as Meso Scale Discovery (MSD) arrays, proximity extension assays such as Olink, microfluidic arrays, homogenous or heterogenous immunoassay analysers such as Olympus AU400 and liquid chromatography-mass spectrometry (LC-MS).

In other embodiments, the biomarkers are detected and measured using ELISA, Western Blot, or flow cytometry. In further embodiments, point-of-care systems such as microfluidic devices or lateral flow assay are used to the detect and measure the biomarkers. However, it is to be appreciated that other techniques can be used to detect and measure biomarker concentrations.

With reference to table 1, in undiluted samples, a concentration of CRP within a range of 160,000 ng/ml to 8,000,000 ng/ml is indicative of bacterial infection. A concentration of neutrophil elastase within a range of 23,000 to 720,000 ng/ml is indicative of bacterial infection. A concentration of β-Defensin 2 within a range of 7,600 to 30,000 ng/ml is indicative of bacterial infection. A concentration of IL-8 within a range of 35,000 to 800,000 ng/ml is indicative of bacterial infection. A concentration of MPO within a range of 20,000,000 to 200,000,000 ng/ml is indicative of bacterial infection. A concentration of TREM-2 within a range of 3,000 to 360,000 ng/ml is indicative of bacterial infection. A concentration of G-CSF within a range of 4,300 to 50,000 ng/ml is indicative of bacterial infection. A concentration of SAA within a range of 45,000 to 300,000 ng/ml is indicative of bacterial infection.

Furthermore, with reference to table 1, concentration of IP-10 within a range of 50 to 800 ng/ml is indicative of viral infection. A concentration of MxA within a range of 0.6 to 320 ng/ml is indicative of viral infection. A concentration of TRAIL within a range of 3,000 to 22,000 ng/ml is indicative of viral infection.

In further embodiments, two or more biomarkers in combination are tested to indicate the cause as either a bacterial infection or viral infection. For example and with reference to table 2, the combination of IP-10 and CRP has 100% sensitivity and 93% specificity.

### Establishing Reference Diagnostic Values

In yet another aspect, the present invention relates to a method for establishing reference diagnostic values to assist with sinonasal inflammatory profiling. The method involves: a) collecting biological samples from a cohort of patients; b) detecting and measuring the concentration of biomarkers in the biological samples that are associated with viral infections and bacterial infections; and c) computing reference diagnostic values based on the measured concentrations. The cohort preferably comprises healthy (control) patients, patients with viral infection, patients with bacterial infections. In embodiments, the cohort comprises patients with mixed infections, however, it is to be appreciated that cohorts without mixed infections can be preferable.

Preferably, the biological samples are sinonasal samples including sinonasal secretions, sinonasal aspirates and sinonasal tissue cultures. Preferably, the sinonasal sample is collected by forced exhalation or absorptive strips. In embodiments, the dilution ratio of the sinonasal sample is a standardised dilution ratio for accurate testing.

The biomarkers measured are preferably selected from a biomarker panel comprising Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2).

### Kits

In yet another aspect, the invention relates to a kit for detecting biomarkers associated with sinonasal inflammatory conditions caused by viral and bacterial infections. In one embodiment, the kit comprises one or more agents for detecting protein biomarkers associated with viral and bacterial infections wherein one or more protein biomarkers are selected from Human β-Defensin 2 (BD-2), C-reactive protein (CRP), Eotaxin-2, Eotaxin-3, Granulocyte-Colony Stimulating Factor (G-CSF), Granzyme B, Interleukin-3 (IL-3), Interleukin-5 (IL-5), Interleukin-8 (IL-8), Interleukin-10 (IL-10), Interleukin-37 (IL-37), Interferon gamma inducible protein-10 (IP-10), Matrix metalloproteinase-1 (MMP-1), Matrix metalloproteinase-8 (MMP-8), Myeloperoxidase (MPO), Human Myxovirus Resistance protein 1 (MxA), Neutrophil Elastase (NE), Serum Amyloid A (SAA), Tumor necrosis factor A (TNFa), Tumor necrosis factor-related apoptosis-inducing ligand (TRAIL), Triggering receptor expressed on myeloid cells-1 (TREM-1), and Triggering receptor expressed on myeloid cells-2 (TREM-2). For example, the one or more agents are antibodies appropriate for detecting proteins using an immunoassay, a colorimetric assay, or a turbidimetric assay.

In another embodiment, the kit comprises one or more agents for detecting nucleic acid biomarkers associated with viral and bacterial infections. For example, the one or more agents are appropriate for detecting RNA using hybridisation techniques and microarrays.

### Experimental Data

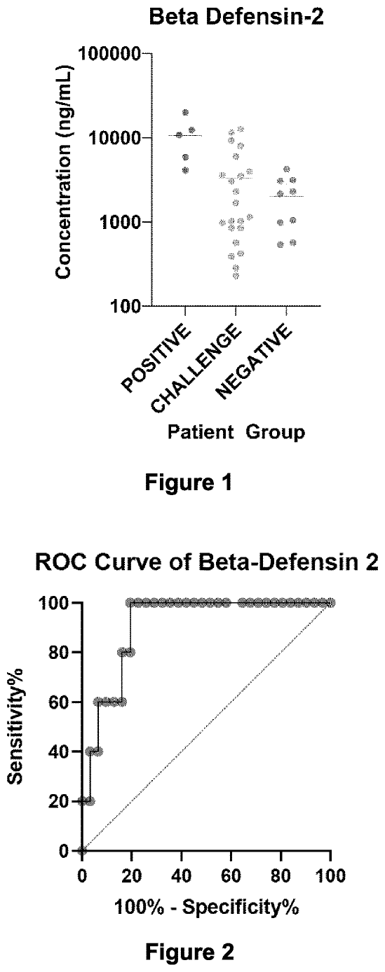

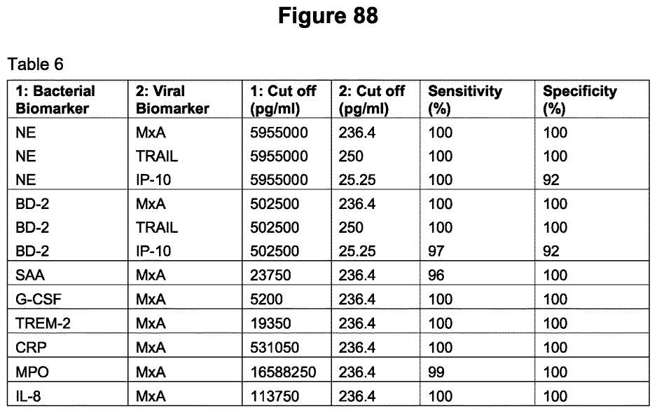

The data contained in FIGS. 1 to 88 was obtained by the following method.

### 1 Method

### 1.1 Collection of Sinonasal Samples

Patients were selected based on a clinical diagnosis confirmed by Polymerase Chain Reaction (PCR), patient symptoms, and clinical history including allergy, disease, and medication history.

Sinonasal samples were collected from patients diagnosed with sinonasal inflammatory conditions through forced exhalation of nasal secretions and from healthy patients using absorptive strips. Collected sinonasal samples were stored at about minus 80 °C under appropriate conditions prior to biomarker quantification.

### 1.2 Quantification of Protein Biomarkers

### 1.2.1 Quantification of Protein Biomarkers Using Luminex Multiplex Assay

The collected samples were incubated at room temperature until thawed. The thawed samples were transferred to 2 ml microcentrifuge tubes with 1000 μl of phosphate-buffered saline (PBS), 0.05% detergent and 1% Bovine Serum Albumin (BSA). The samples were gently vortexed for 30 s, syringe-filtered 15 times and incubated for 30 min at 4 °C. Following incubation, the samples were centrifuged for 25 min at 16,000 g at 4 °C. The supernatant was pipetted into a new microcentrifuge tube and diluted 1:2 with Luminex buffer. The concentrations of the protein biomarkers including BD-2, CRP, Eotaxin-2, Eotaxin-3, G-CSF, Granzyme B, IL-3, IL-5, IL-8, IL-10, IL-37, IP-10, MMP-1, MMP-8, MPO, MxA, NE, SAA, TNFa, TRAIL, TREM-1, and TREM-2 were measured using Luminex multiplex assay as per the manufacturer's instructions.

### 1.2.2 Quantification of Protein Biomarkers Using Elisa

A sandwich enzyme immunoassay kit by BioVendor (Lot: E21-049) was used to detect and quantify the concentration of Human MxA protein in the collected sinonasal samples. Reagents were brought to room temperature prior to use. The master standard was reconstituted with dilution buffer and gently agitated for 15 min. The master standard was aliquoted in duplicates into a 96-well microtiter plate at concentrations of 6 ng/ml, 3ng/ml, 1.5 ng/ml, 0.75 ng/ml and 0.375 ng/ml and at a final volume of 100 ml. Sinonasal samples collected from each patient group were aliquoted in duplicates into the microtiter plate at different dilutions. The microtiter plate was incubated at room temperature for 1 hour at 300 RPM. The wells were washed 3 times with the wash solution and the plate was inverted and tapped against a paper towel to remove excess solution. One hundred microlitres of Biotin Labelled Anti-MxA Antibody solution was pipetted into each well and the plate was incubated at room temperature for 1 hour at 300 RPM. The wells were washed again 3 times with 350 ml of the wash solution, inverted and tapped against a paper towel to remove excess solution. One hundred microlitres of Streptavidin-HRP conjugate was pipetted into each well and the plate was incubated for 1 hour at room temperature at 300 RPM. The wells were washed 5 times with 350 ml of wash solution and 100 ml of substrate solution was pipetted into each well. The plate was incubated for 20 minutes at room temperature. One hundred microlitres of stop solution was pipetted into each well to stop the enzymatic reaction and optical density was measured at 450/630 nm.

A sandwich enzyme immunoassay kit by Alpha Diagnostic (Cat: 100-250-BD2) was used to detect and quantify the concentration of β-Defensin 2 protein in the collected sinonasal samples. Reagents were brought to room temperature prior to use and reconstituted and diluted as per manufacturer's instructions. One hundred microlitres of each collected sample, controls and standards were aliquoted in duplicates into a microtiter plate and incubated for 60 min at room temperature. The wells were washed 4 times with wash solution and excess liquid was decanted onto a paper towel after the final wash. One hundred microlitres of Anti-BD-2 Detecting Antibody was pipetted into each well and the plate was incubated at room temperature for 60 min. The wells were washed again 4 times as previously described. One hundred microlitres of Streptavidin-HRP Conjugate was pipetted into each well and the plate was incubated for 30 min at room temperature. The well were washed 5 times as previously described. One hundred microlitres of TMB Substrate was pipetted into each well and the plate was incubated for 15 min in the dark. One hundred microlitres of Stop Solution was pipetted into each well and the plate was gently mixed. Optical density was read at 450/630 nm.

A sandwich enzyme immunoassay kit by Hycult Biotech (Lot: 27909K0221-K) was used to detect and quantify the concentration of Neutrophil Elastase protein in the collected sinonasal samples. Reagents were brought to room temperature prior to use and reconstituted and diluted as per manufacturer's instructions. One hundred microlitres of each collected sample, controls and standards were aliquoted in duplicates into a microtiter plate and incubated for 1 hour at room temperature. The wells were washed 4 times with wash buffer and excess liquid was decanted onto a paper towel after the final wash. Two hundred microlitres of wash buffer was pipetted into each well and the plate was incubated for 20 sec at room temperature. Excess solution was decanted, and the wash process was repeated 3 times. One hundred microlitres of diluted tracer was pipetted into each well and the plate was incubated for 1 hour at room temperature. The wash process was repeated as previously described. One hundred microlitres of diluted streptavidin-peroxidase was pipetted into each well and the plate was incubated for 1 hour at room temperature. The wash process was repeated as previously described. One hundred microlitres of TMB substrate was pipetted into each well and the plate was incubated for 30 min at room temperature. One hundred microlitres of stop solution was pipetted into each well and the plate was agitated to mix the solutions. Optical density was read at 450 nm for 30 min.

### 1.3 Calculating Reference Diagnostic Values

Reference diagnostic values for sinonasal inflammatory conditions caused by viral and bacterial infections were established by inputting the measured concentrations into GraphPad Prism and generating receiver operating characteristic (ROC) curves.

The ROC curve is used to generate three values of importance: Min corresponding to lowest sensitivity and maximum specificity, Max corresponding to highest sensitivity and lowest specificity, and Peak corresponding to balance sensitivity and specificity. A Min cutoff, Max cutoff, and Peak cutoff values are determined corresponding to the biomarker concentration at Min, Max, and Peak, respectively.

For combinations of bacterial and viral biomarkers, the sensitivity and specificity between bacterial and viral biomarkers are calculated using the following equations: Sensitivity=(Sensitivity A+Sensitivity B)−(Sensitivity A×Sensitivity B) and Specificity=(Specificity A×Specificity B), where A is bacterial biomarker and B is viral biomarker.

### Study 1

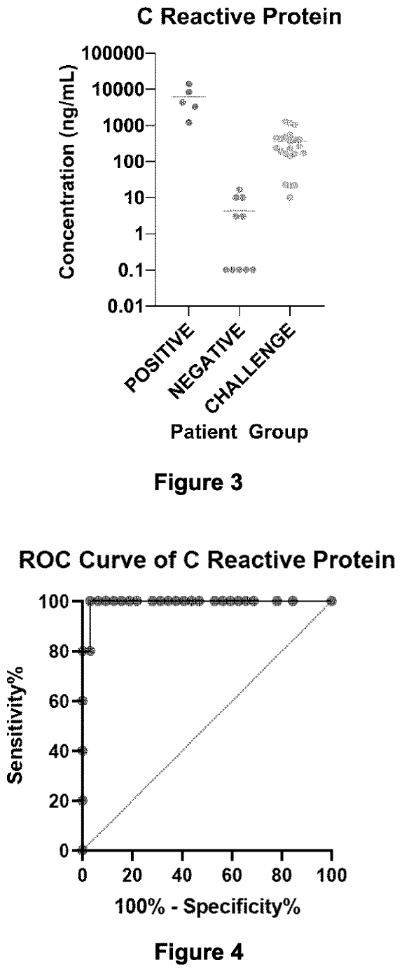

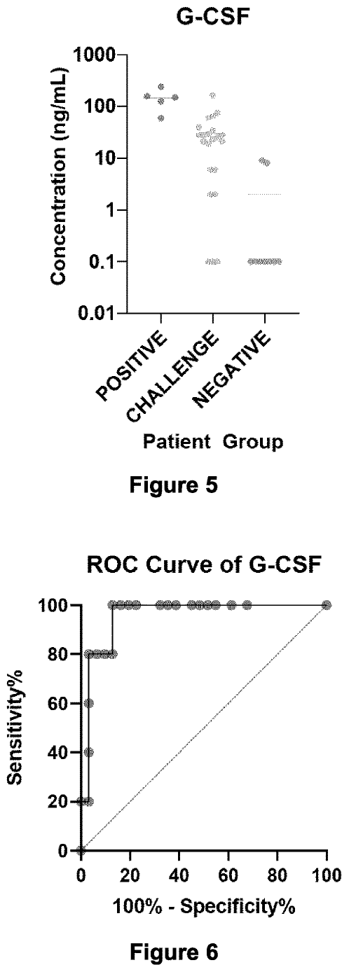

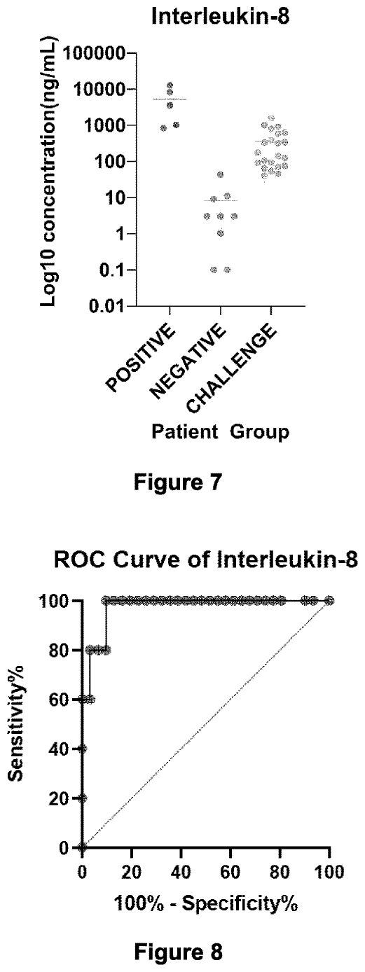

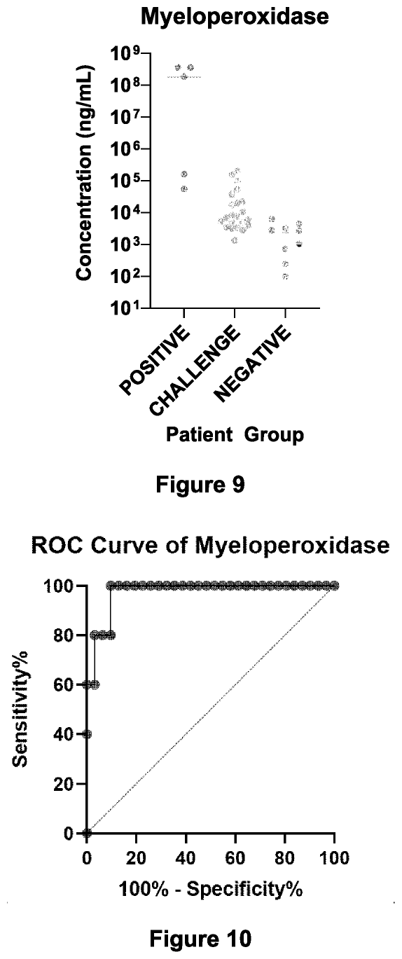

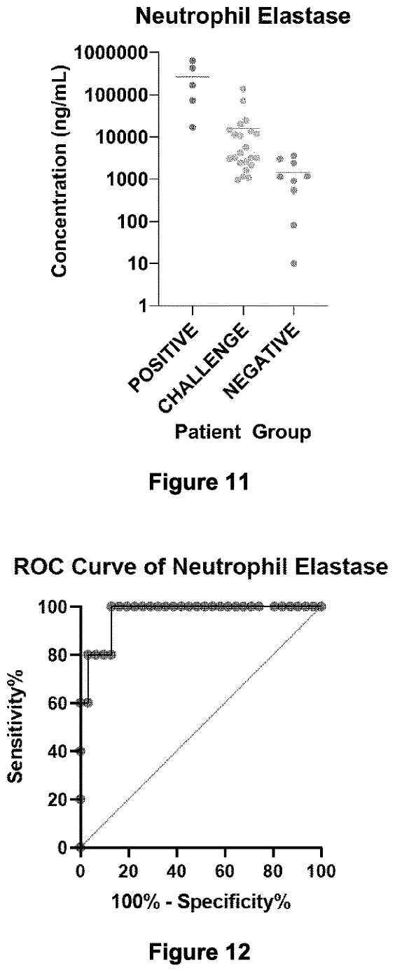

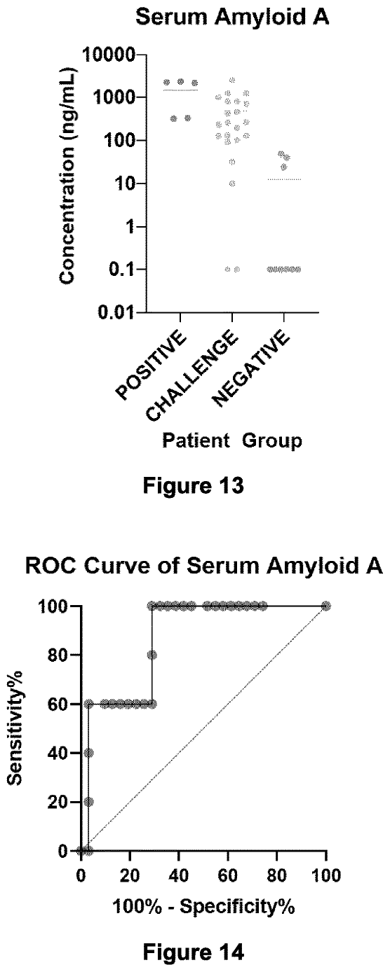

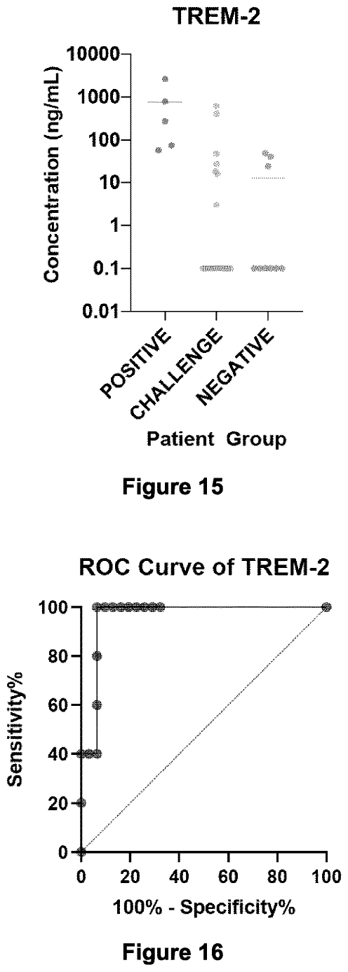

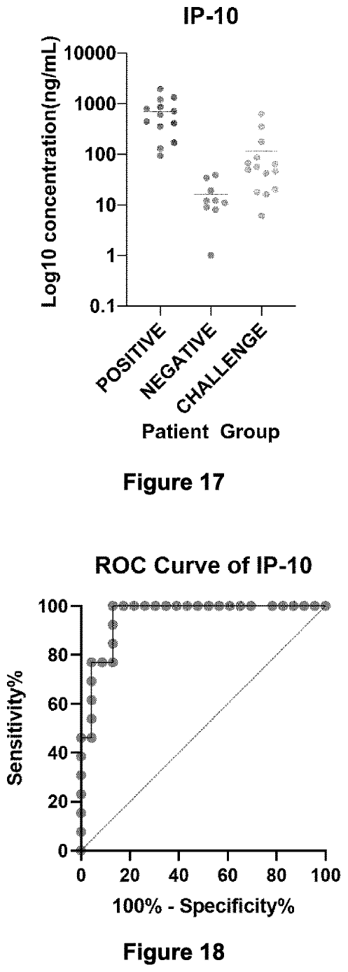

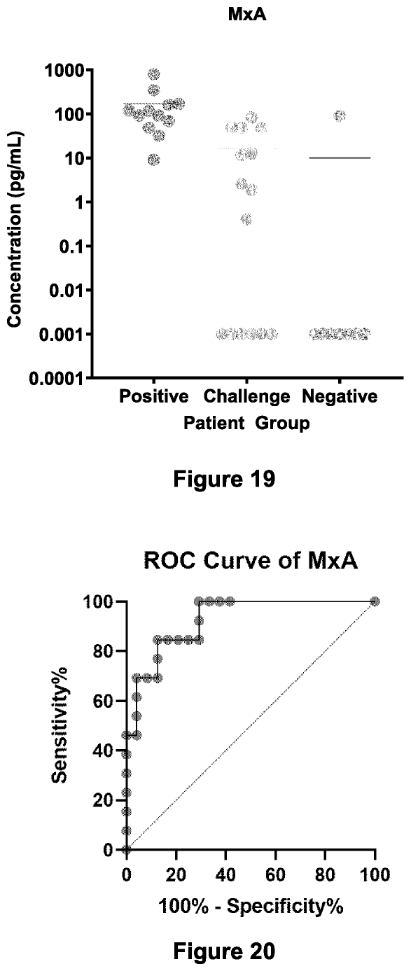

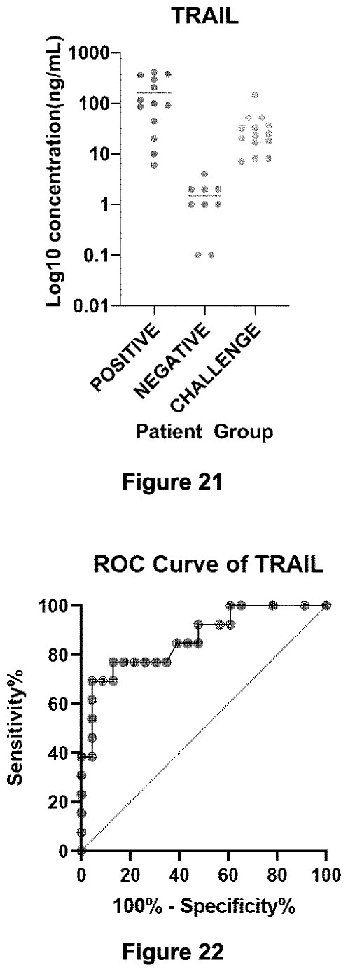

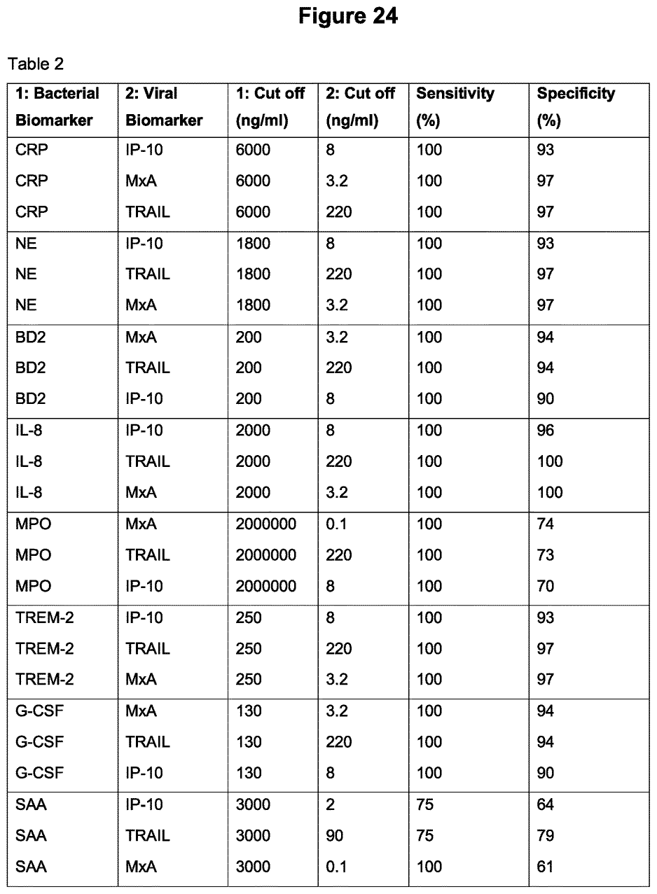

In a first study, sinonasal samples were collected from 28 patients diagnosed with nasal inflammatory conditions and 9 healthy patients. Concentrations of the biomarkers including bacterial biomarkers BD-2 (FIG. 1), CRP (FIG. 3), G-CSF (FIG. 5), IL-8 (FIG. 7), MPO (FIG. 9), NE (FIG. 11), SAA (FIG. 13), and TREM-2 (FIG. 15) and viral biomarkers IP-10 (FIG. 17), MxA (FIG. 19), and TRAIL (FIG. 21) were measured using Luminex multiplex assay or ELISA. ROC curves (FIGS. 2, 4, 6, 8, 10, 12, 14, 16, 18, 20, 22) were generated and reference diagnostic range thresholds including cut off values, associated sensitivity and specificity values, accuracy, area under the curve (AUC), 95% confidence interval and P-value (FIG. 23) were determined from the ROC curves. For example and with reference to FIG. 23, BD-2, CRP, G-CSF, IL-8, NE, TREM-2, at peak cutoff threshold, all had sensitivity, specificity, and accuracy above 90%. Combinations of two biomarkers were also tested to indicate the cause as either a bacterial infection or viral infection and the reference diagnostic range thresholds are shown in FIG. 24. For example and with reference to FIG. 24, the combination of IP-10 and CRP has 100% sensitivity and 93% specificity at the specified cutoff concentrations.

### Study 2

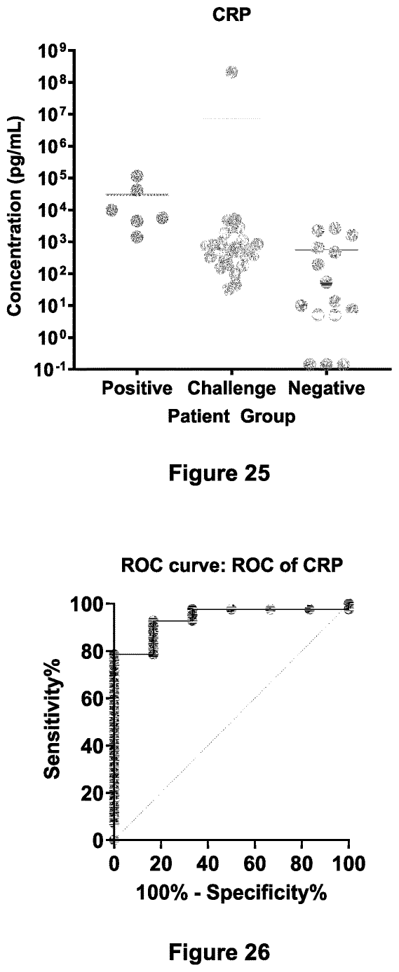

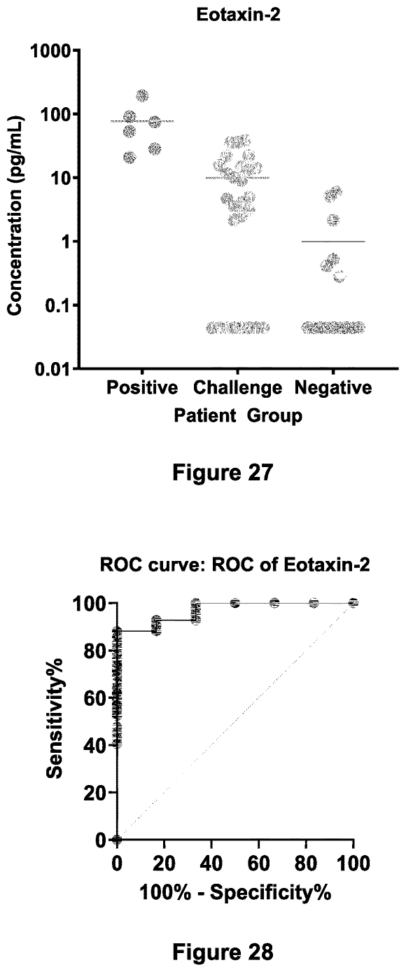

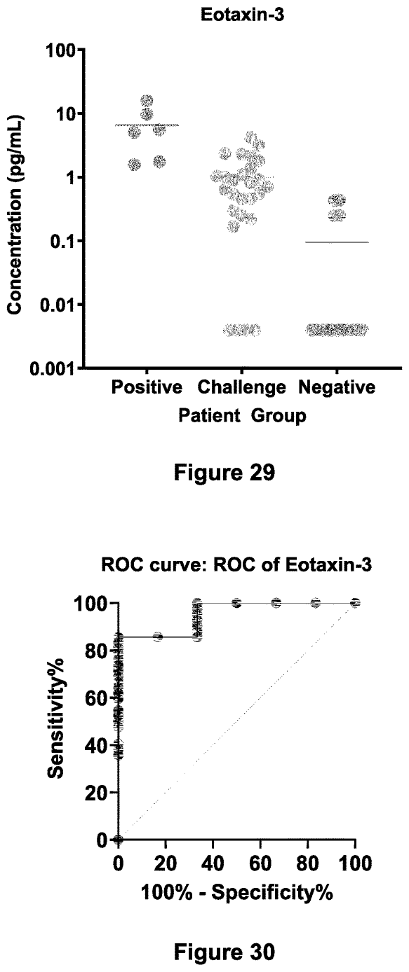

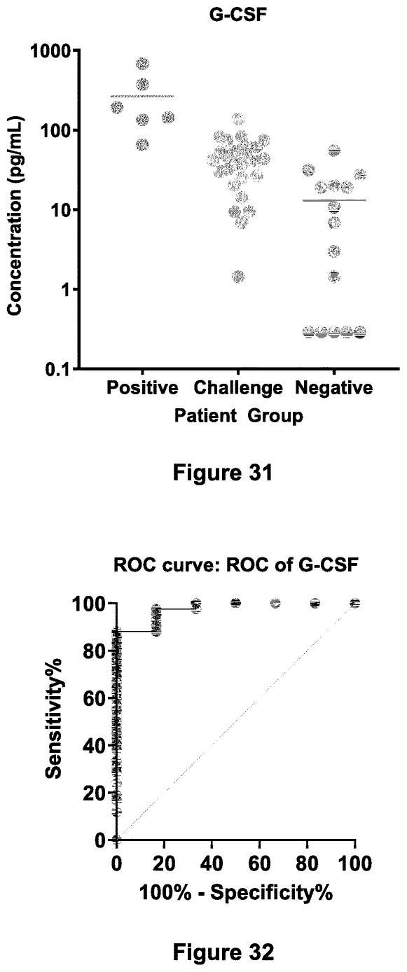

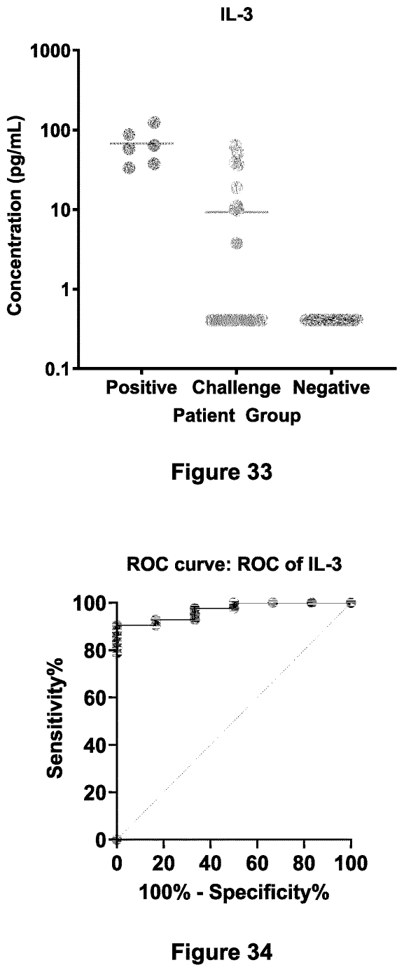

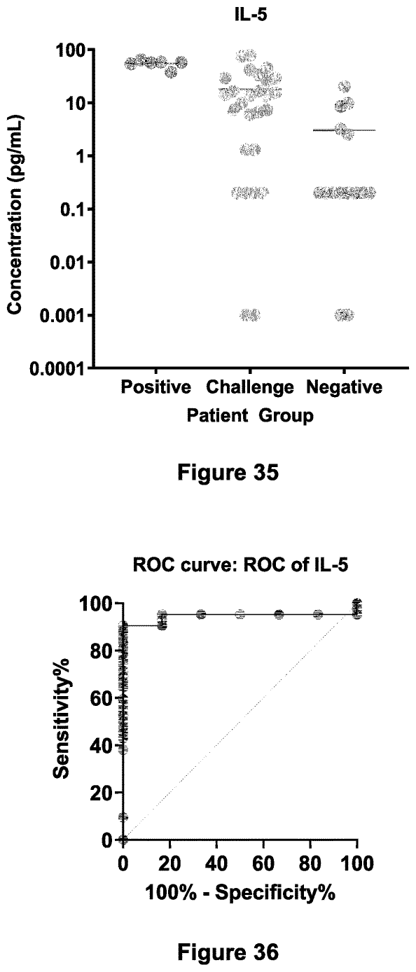

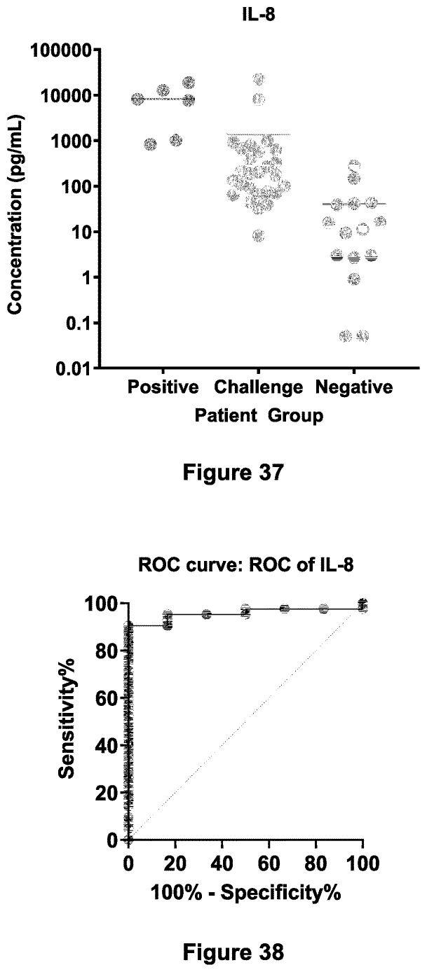

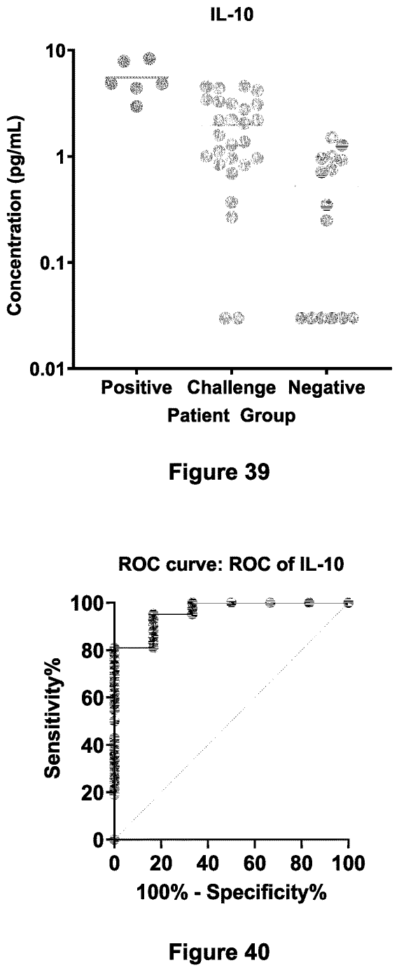

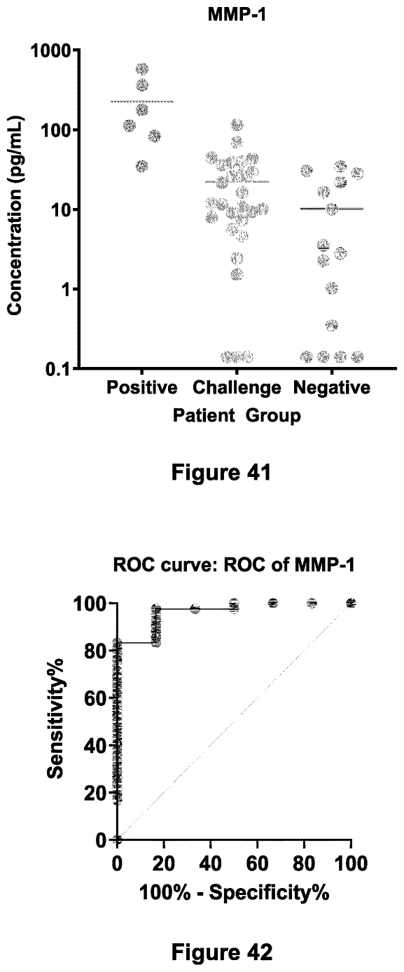

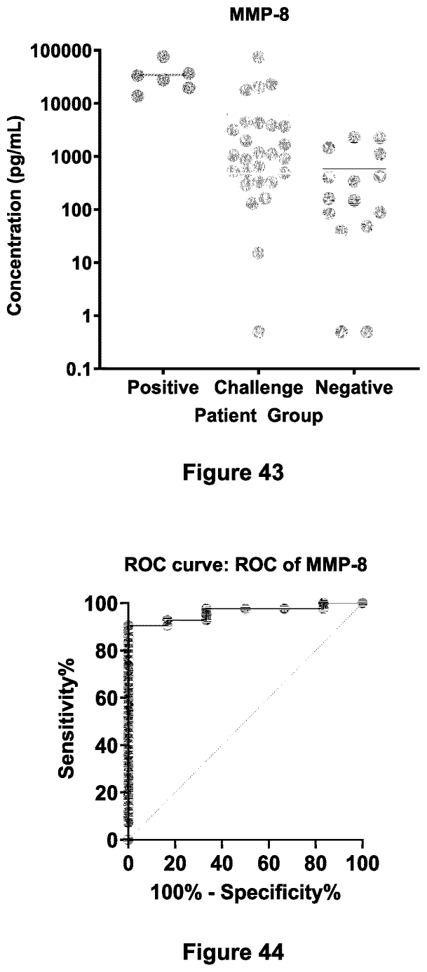

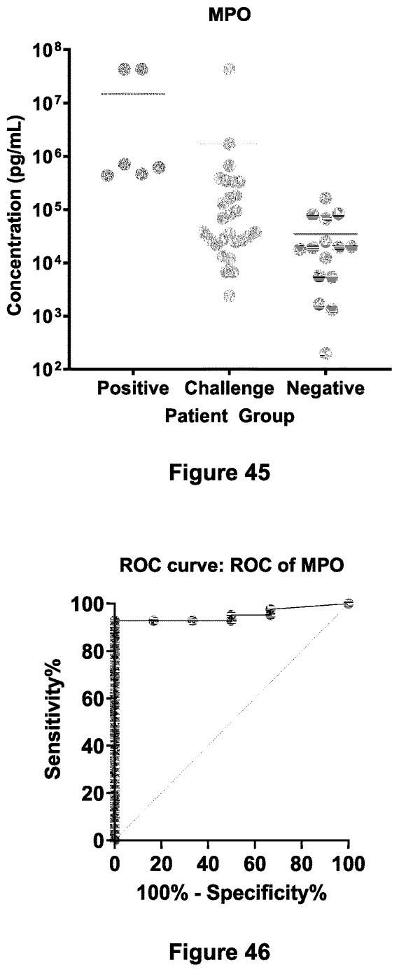

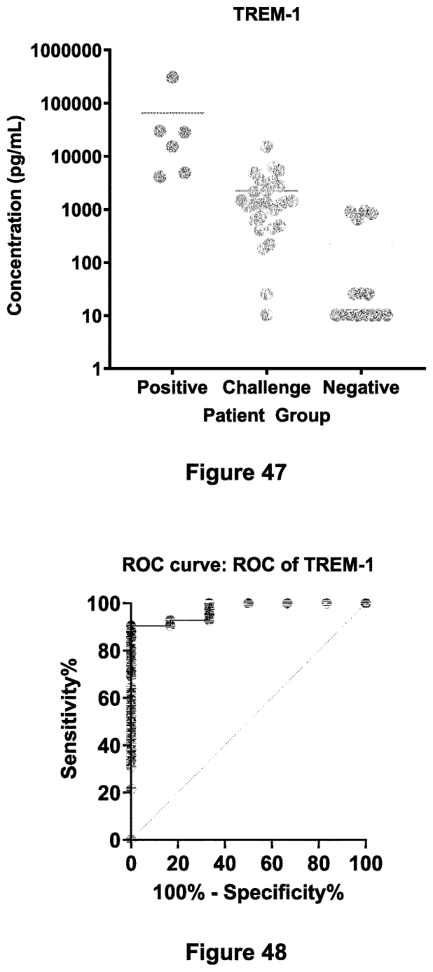

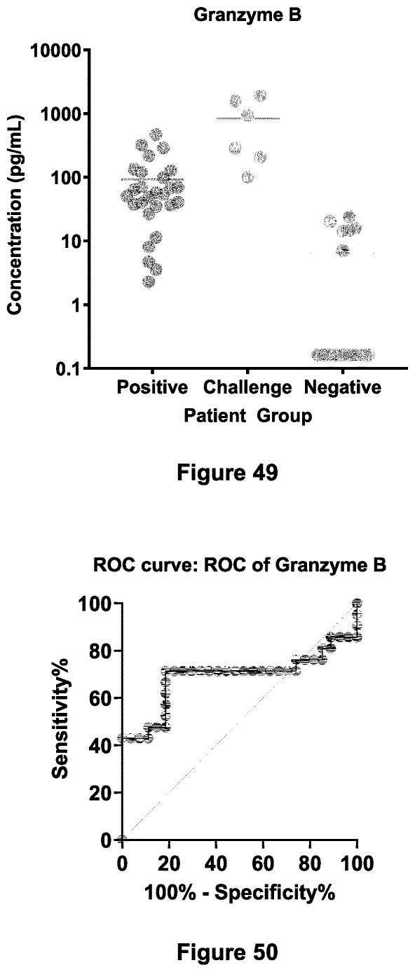

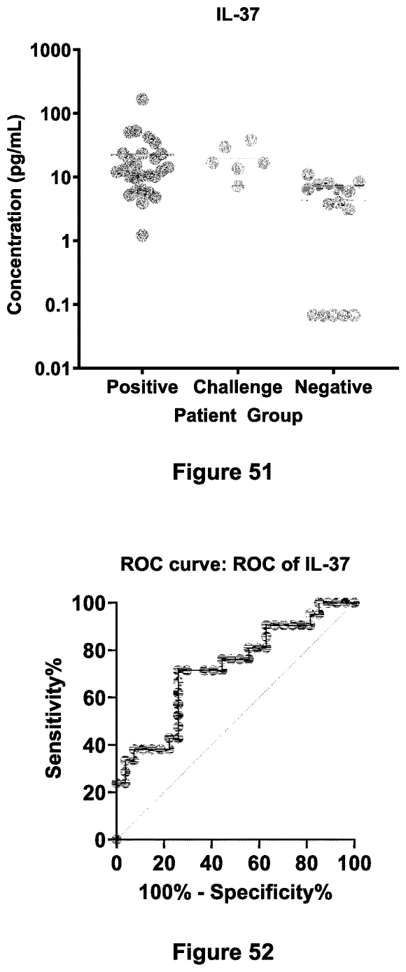

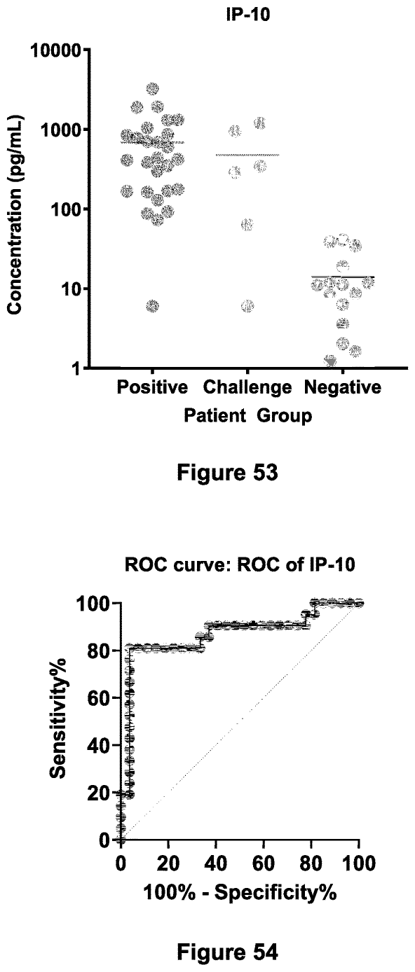

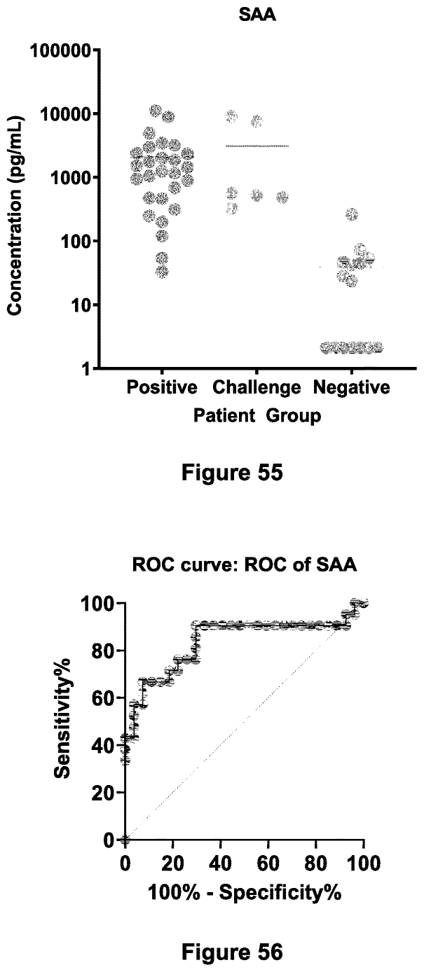

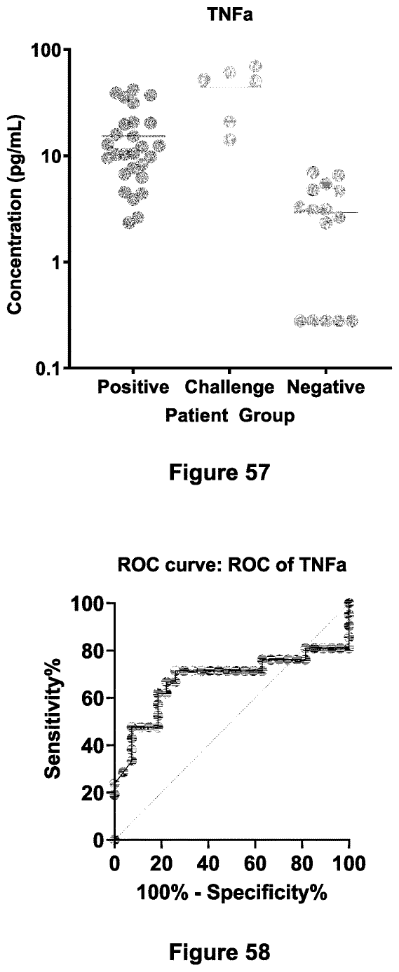

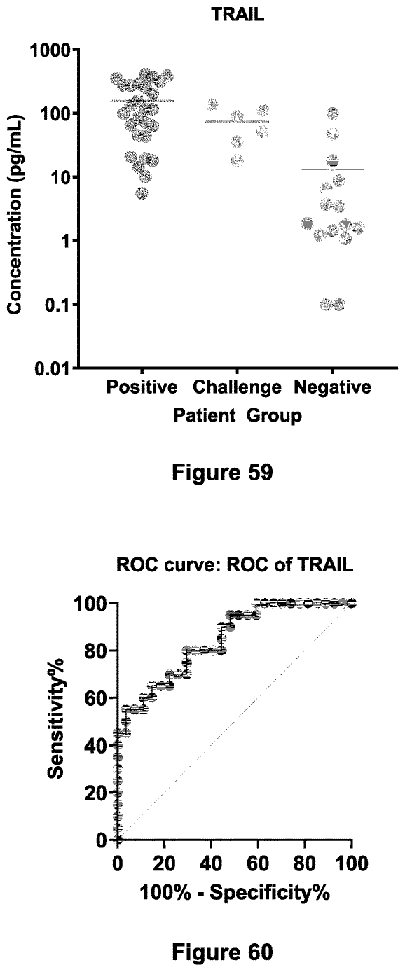

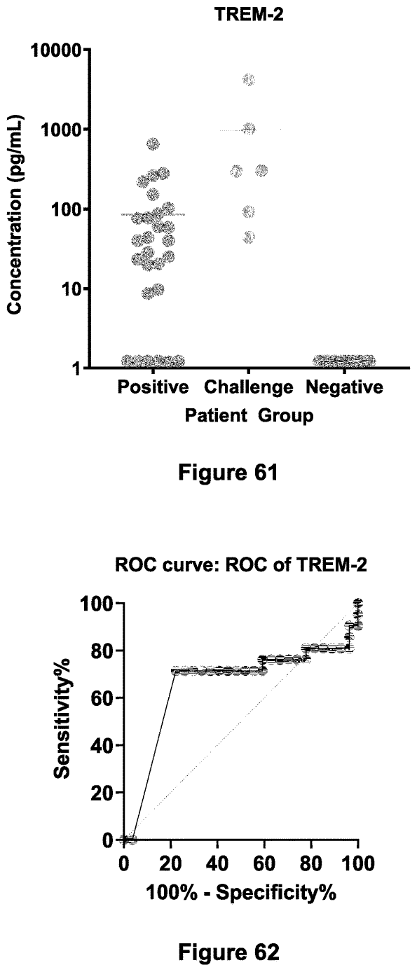

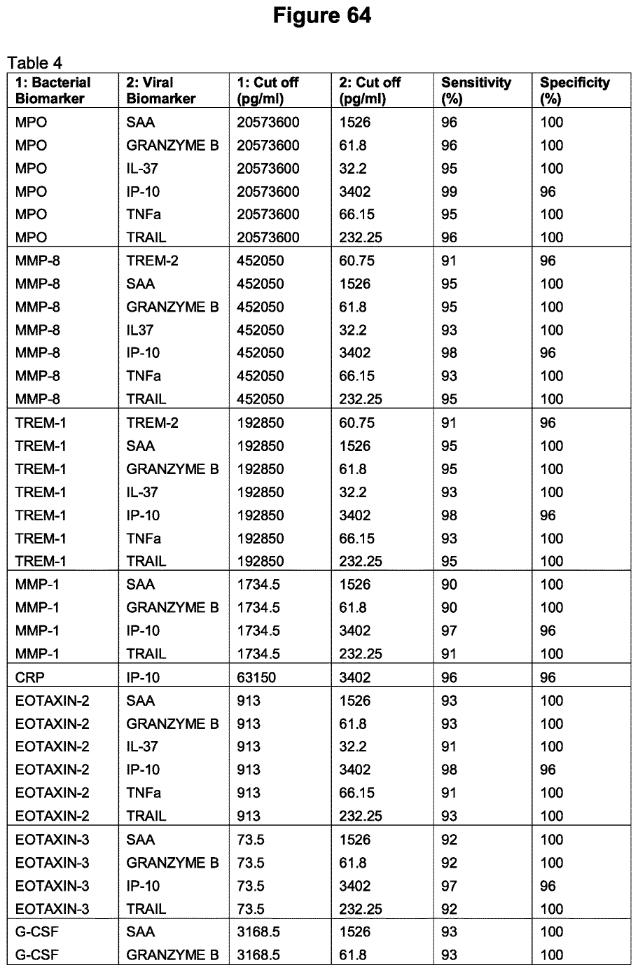

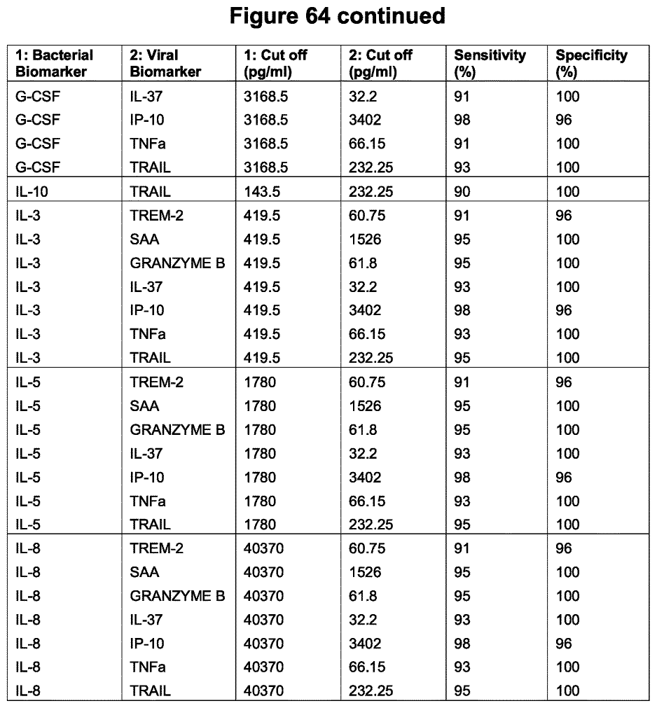

In a second study, sinonasal samples were collected from 69 patients. After excluding 21 patients who had some form of allergy and 1 patient diagnosed with both viral and bacterial infection, 47 samples remained, wherein 6 had bacterial infection, 26 had viral infection, and 15 were healthy. Concentrations of the biomarkers including bacterial biomarkers CRP (FIG. 25), Eotaxin-2 (FIG. 27), Eotaxin-3 (FIG. 29), G-CSF (FIG. 31), IL-3 (FIG. 33), IL-5 (FIG. 35), IL-8 (FIG. 37), IL-10 (FIG. 39), MMP-1 (FIG. 41), MMP-8 (FIG. 43), MPO (FIG. 45), and TREM-1 (FIG. 47), and viral biomarkers Granzyme B (FIG. 49), IL-37 (FIG. 51), IP-10 (FIG. 53), SAA (FIG. 55), TNFa (FIG. 57), TRAIL (FIG. 59), TREM-2 (FIG. 61) were measured using Luminex multiplex assay. ROC curves (FIGS. 26, 28, 30, 32, 34, 36, 38, 40, 42, 44, 46, 48, 50, 52, 54, 56, 58, 60, 62) were generated and reference diagnostic range thresholds including cut off values, associated sensitivity and specificity values, area under the curve (AUC), 95% confidence interval and P-value (FIG. 63) were determined from the ROC curves. Combinations of two biomarkers were also tested to indicate the cause as either a bacterial infection or viral infection and the reference diagnostic range thresholds are shown in FIG. 64.

### Study 3

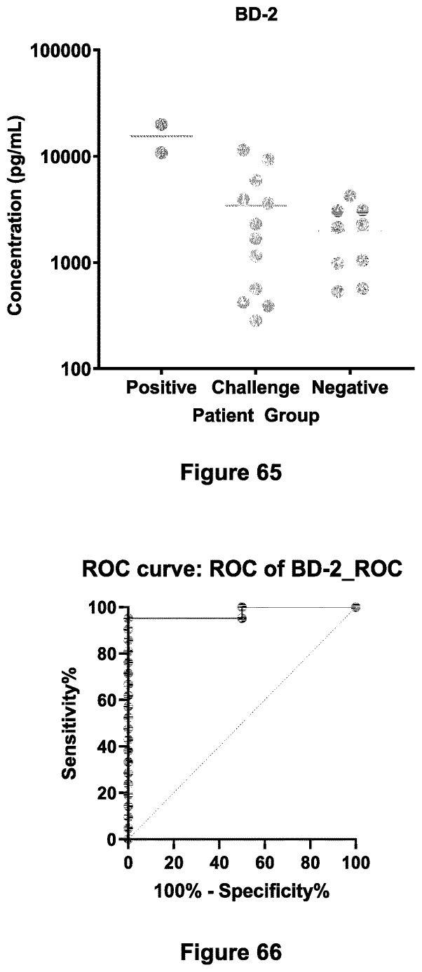

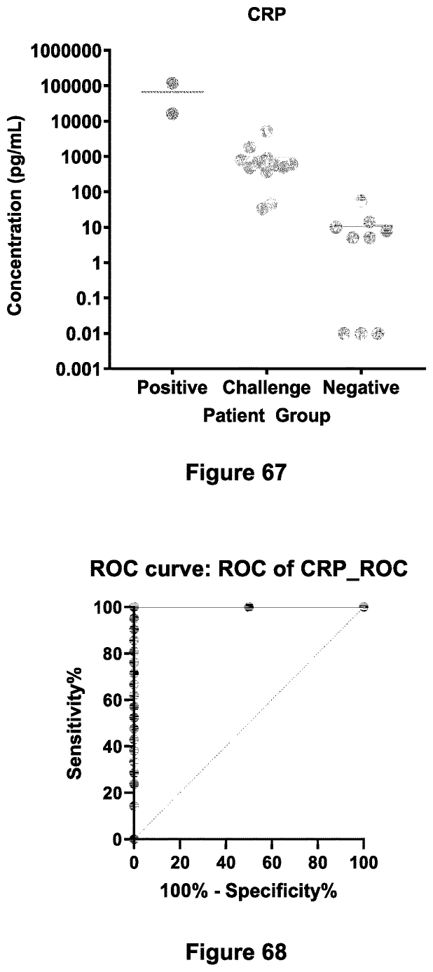

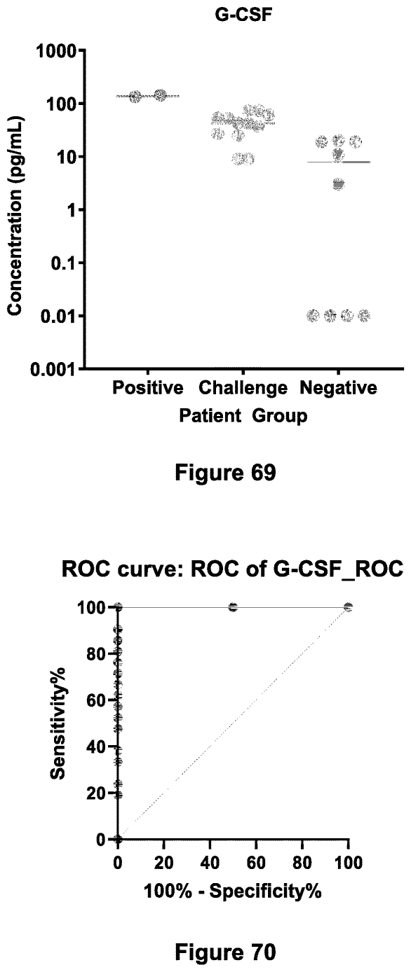

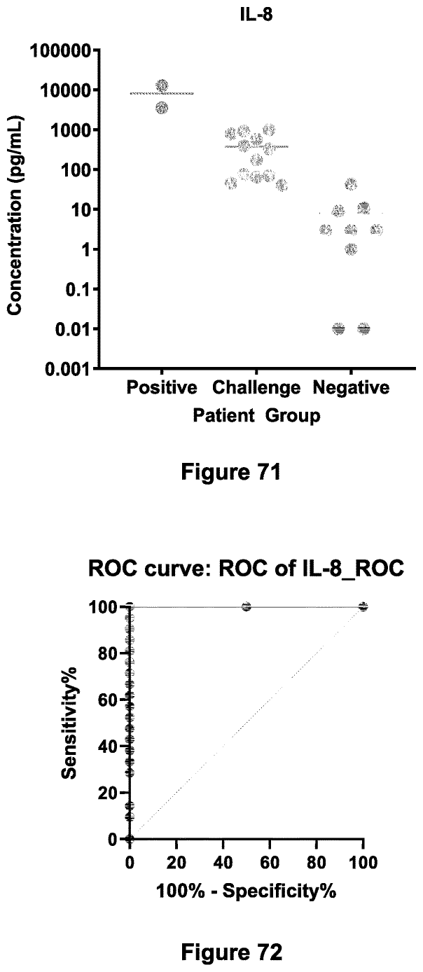

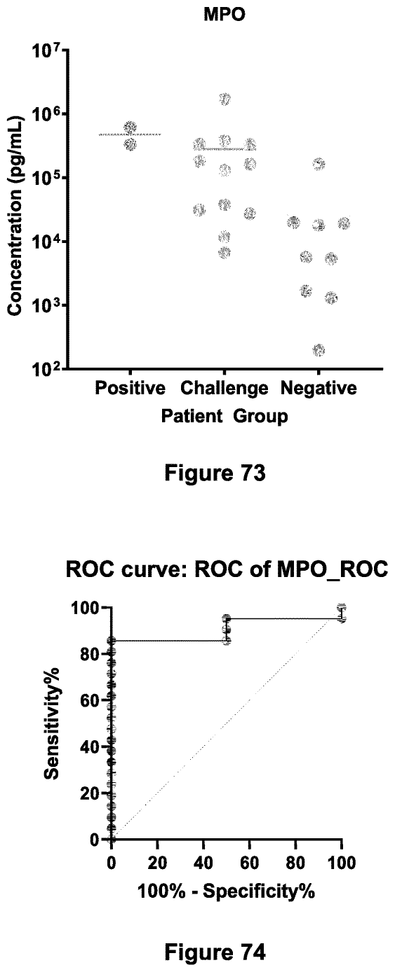

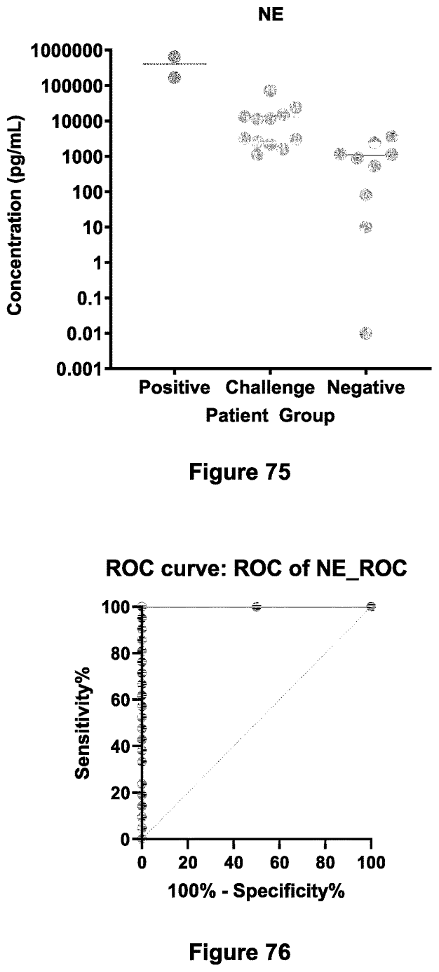

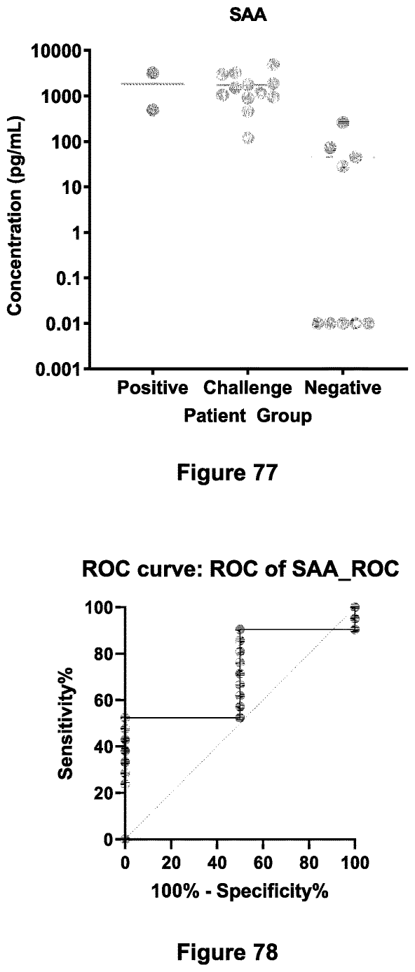

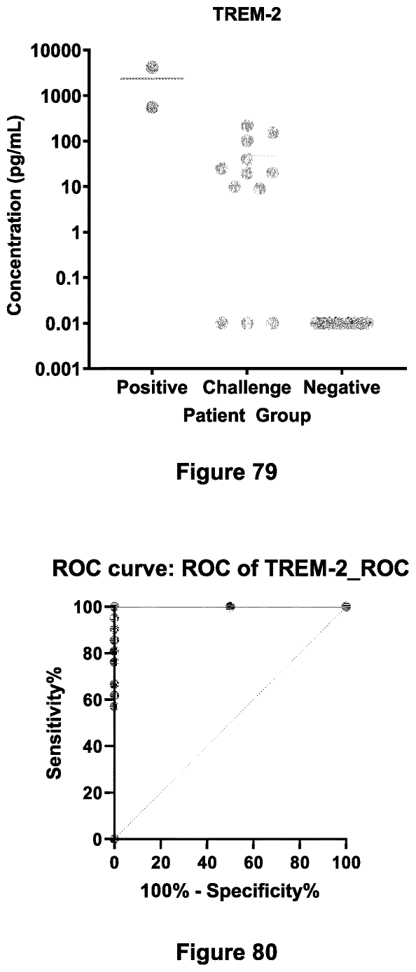

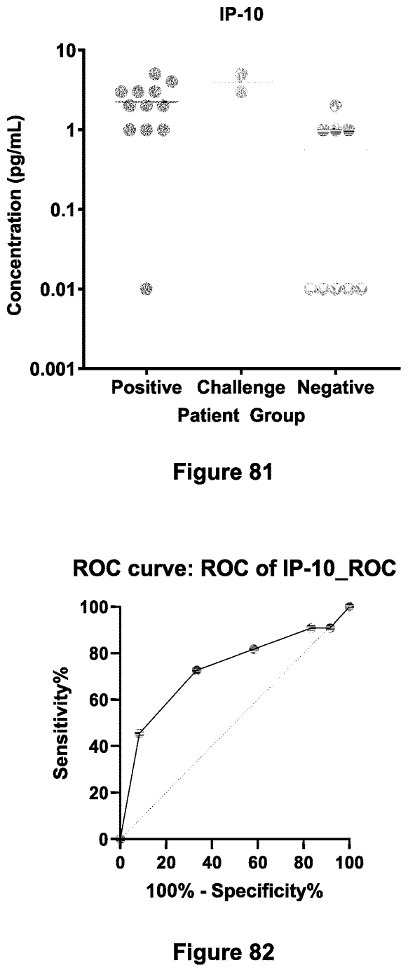

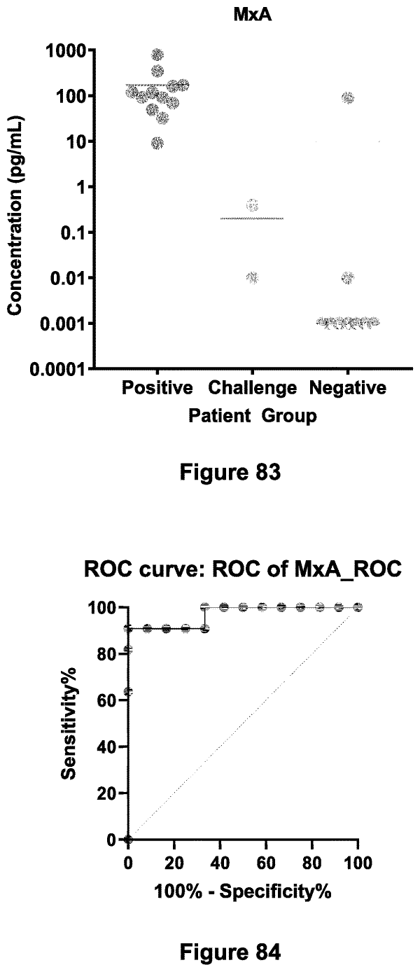

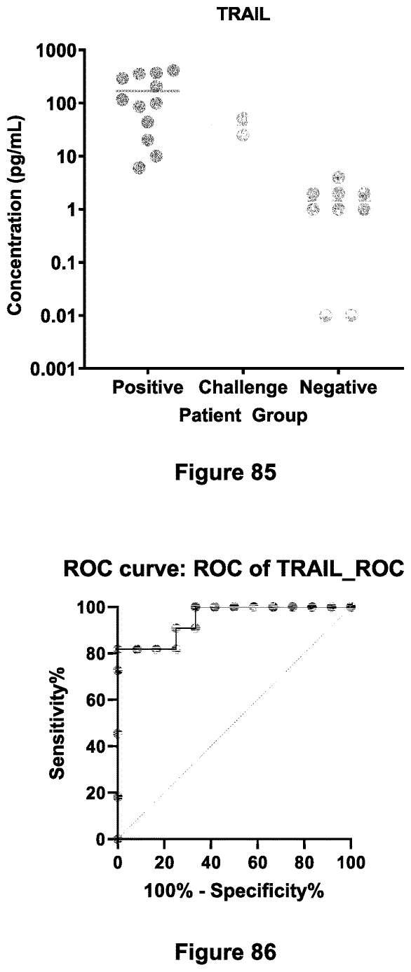

In a third study, sinonasal samples were collected from 36 patients. After excluding 12 patients who had some form of allergy and 1 patient diagnosed with both viral and bacterial infection, 23 samples remained, wherein 2 had bacterial infection, 12 had viral infection, and 9 were healthy. Concentrations of the biomarkers including bacterial biomarkers BD-2 (FIG. 65), CRP (FIG. 67), G-CSF (FIG. 69), IL-8 (FIG. 71), MPO (FIG. 73), NE (FIG. 75), SAA (FIG. 77), and TREM-2 (FIG. 79) and viral biomarkers IP-10 (FIG. 81), MxA (FIG. 83), TRAIL (FIG. 85) were measured using Luminex multiplex assay or ELISA. ROC curves (FIGS. 66, 68, 70, 72, 74, 76, 78, 80, 82, 84, 86) were generated and reference diagnostic range thresholds including cut off values, associated sensitivity and specificity values, area under the curve (AUC), 95% confidence interval and P-value (FIG. 87) were determined from the ROC curves. Combinations of two biomarkers were also tested to indicate the cause as either a bacterial infection or viral infection and the reference diagnostic range thresholds are shown in FIG. 88.

For the avoidance of doubt, the results of Study 1 demonstrate that SAA and TREM-2 are suitable as bacterial biomarkers, whereas the results of Study 2 demonstrate that SAA and TREM-2 are suitable as viral biomarkers. Accordingly, the results of the Studies support embodiments in which SAA and TREM-2 are bacterial biomarkers only and support embodiments in which SAA and TREM-2 are viral biomarkers only or embodiments where SAA and TREM-2 are bacterial or viral biomarkers.

1. A method for treating a sinonasal inflammatory condition in a patient caused by a viral or bacterial infection comprising:
administering a therapeutically effective amount of an antibiotic or an antiviral agent to the patient.
wherein a concentration of a first biomarker in a sinonasal sample from the patient is within a reference diagnostic range threshold for the first biomarker or a concentration of a second biomarker in the sinonasal sample from the patient is within a reference diagnostic range threshold for the second biomarker,
wherein the first biomarker is a protein and the second biomarker is a protein,
wherein the first biomarker is selected from a panel of biomarkers for detection of a viral cause of sinonasal inflammation and the second biomarker is selected from a panel of biomarkers for detection of a bacterial cause of sinonasal inflammation,
wherein the biomarkers for detection of a viral cause of sinonasal inflammation include Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2) and the biomarkers for detection of a bacterial cause of sinonasal inflammation include Human β-Defensin 2 (BD-2), C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-International

8. Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1 (TREM-1), and Triggering receptor expressed on myeloid cells 2 (TREM-2), and
wherein a concentration of the first biomarker within the reference diagnostic range threshold for the first biomarker is indicative of a viral infection and a concentration of the second biomarker within the reference diagnostic range threshold for the second biomarker is indicative of a bacterial infection.

2. The method of claim 1, wherein the first biomarker is any one of Triggering receptor expressed on myeloid cells 2 (TREM-2), Serum amyloid A (SAA), Granzyme B, Interleukin-37 (IL-37), Interferon gamma inducible protein-10 (IP-10), Tumor necrosis factor A (TNFa), and Tumour Necrosis Factor-Related Apoptosis-Inducing Ligand (TRAIL), and the second biomarker is any one of Interleukin-3 (IL-3), Interleukin-5 (IL-5) Matrix metalloproteinase-8 (MMP-8) and Triggering receptor expressed on myeloid cells-1 (TREM-1).

3. The method of claim 1, wherein the first biomarker is any one of Serum amyloid A (SAA), Granzyme B, Interleukin-37 (IL-37), Interferon gamma inducible protein-10 (IP-10), Tumor necrosis factor A (TNFa), Tumour Necrosis Factor-Related Apoptosis-Inducing Ligand (TRAIL), and Human Myxovirus Resistance Protein 1 (MxA), and the second biomarker is any one of Granulocyte colony stimulating factor (G-CSF) and Myeloperoxidase (MPO).

4. The method of claim 1, wherein the first biomarker is any one of Serum amyloid A (SAA), Granzyme B, Interferon gamma inducible protein-10 (IP-10), and Tumor necrosis factor A (TNFa), and the second biomarker is any one of Eotaxin-3 and Matrix metalloproteinase-1 (MMP-1).

5. The method of claim 1, wherein the first biomarker is any one of Human Myxovirus Resistance Protein 1 (MxA), Tumour Necrosis Factor-Related Apoptosis-Inducing Ligand (TRAIL), and Interferon gamma inducible protein-10 (IP-10), and the second biomarker is any one of Human-Defensin 2 (BD-2) and Neutrophil Elastase (NE).

6. The method of claim 1, wherein the first biomarker is any one of Serum amyloid A (SAA), Granzyme B, Interleukin-37 (IL-37), Interferon gamma inducible protein-10 (IP-10), Tumor necrosis factor A (TNFa), and Tumour Necrosis Factor-Related Apoptosis-Inducing Ligand (TRAIL), and the second biomarker is Eotaxin-2.

7. The method of claim 1, wherein the first biomarker is any one of Triggering receptor expressed on myeloid cells 2 (TREM-2) and Serum amyloid A (SAA), and the second biomarker is Human Myxovirus Resistance Protein 1 (MxA).

8. The method of claim 1, wherein the first biomarker is any one of Triggering receptor expressed on myeloid cells 2 (TREM-2), Serum amyloid A (SAA), Granzyme B, Interleukin-37 (IL-37), Interferon gamma inducible protein-10 (IP-10), Tumor necrosis factor A (TNFa), Tumour Necrosis Factor-Related Apoptosis-Inducing Ligand (TRAIL), and Human Myxovirus Resistance Protein 1 (MxA), and the second biomarker is Interleukin-8 (IL-8).

9. The method of claim 1, wherein the first biomarker is any one of Interferon gamma inducible protein-10 (IP-10) and Human Myxovirus Resistance Protein 1 (MxA), and the second biomarker is C-reactive protein (CRP).

10. The method of claim 1, wherein the first biomarker is Tumour Necrosis Factor-Related Apoptosis-Inducing Ligand (TRAIL), and the second biomarker is Interleukin-10 (IL-10).

11. The method of claim 1, wherein the first biomarker is any one of Human Myxovirus Resistance Protein 1 (MxA) and Interferon gamma inducible protein-10 (IP-10) and the second biomarker is any one of Myeloperoxidase (MPO), Matrix metalloproteinase-8 (MMP-8), and C-Reactive Protein (CRP).

12. The method of claim 1, wherein the first biomarker is Human Myxovirus Resistance Protein 1 (MxA) and the second biomarker is Myeloperoxidase (MPO).

13. The method of claim 1, wherein the first biomarker is Interferon gamma inducible protein-10 (IP-10) and the second biomarker is Myeloperoxidase (MPO).

14. The method of claim 1, wherein the first biomarker is Interferon gamma inducible protein-10 (IP-10) and the second biomarker is Matrix metalloproteinase-8 (MMP-8).

15. The method of claim 1, wherein the first biomarker is Human Myxovirus Resistance Protein 1 (MxA) and the second biomarker is C-Reactive Protein (CRP).

16. The method of claim 1, wherein the sinonasal sample is sinonasal secretions, sinonasal aspirates or tissue cultures.

17. (canceled)

18. A method for treating a sinonasal inflammatory condition in a patient caused by a bacterial and/or viral infection including:
administering a therapeutically effective amount of an antibiotic and/or an antiviral agent, respectively;
monitoring the response of the patient to the treatment including measuring concentrations of a combination of two biomarkers in a sinonasal sample following the administration of the treatment or therapy; and
comparing the measured concentrations of the two biomarkers to a reference diagnostic range thresholds for the two biomarkers; wherein the biomarkers are protein biomarkers selected from a plurality of biomarkers for detection of a viral cause of sinonasal inflammation and the second biomarker is selected from a panel of biomarkers for detection of a bacterial cause of sinonasal inflammation,
wherein the biomarkers for detection of a viral cause of sinonasal inflammation include Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2) and the biomarkers for detection of a bacterial cause of sinonasal inflammation include Human B-Defensin 2 (BD-2), C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-8), Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1 (TREM-1), and Triggering receptor expressed on myeloid cells 2 (TREM-2)
wherein a concentration of the first biomarker within the reference diagnostic range threshold for the first biomarker is indicative of a viral infection and a concentration of the second biomarker within the reference diagnostic range threshold for the second biomarker is indicative of a bacterial infection.

19. (canceled)

20. A sinonasal sample diagnostic kit for diagnosing sinonasal inflammatory conditions caused by viral or bacterial infections from a sinonasal sample collected from a patient, the kit comprising reagents for measuring concentrations of a combination of two protein biomarkers,
wherein the first biomarker is selected from a panel of biomarkers for detection of a viral cause of sinonasal inflammation including Human Myxovirus Resistance Protein 1 (MxA), Granzyme B, Interleukin-37 (IL-37), Interferon gamma inducible protein-10 (IP-10), Serum Amyloid A (SAA), Tumor necrosis factor A (TNFa), Tumour Necrosis Factor-Related Apoptosis-Inducing Ligand (TRAIL), and Triggering receptor expressed on myeloid cells-2 (TREM-2), and the second biomarker is selected from a panel of biomarkers for detection of a bacterial cause of sinonasal inflammation including Human B-Defensin 2 (BD-2), C-Reactive Protein (CRP), Eotaxin-2, Eotaxin-3, Granulocyte colony stimulating factor (G-CSF), Interleukin-3 (IL-3), Interleukin-5 (IL-5), Interleukin-8 (IL-8), Interleukin-10 (IL-10), Myeloperoxidase (MPO), Matrix metalloproteinase-1 (MMP-1), Matrix metalloproteinase-8 (MMP-8), Neutrophil Elastase (NE), and Serum amyloid A (SAA), Triggering receptor expressed on myeloid cells-1 (TREM-1), and Triggering receptor expressed on myeloid cells 2 (TREM-2);
wherein the reagents include antibodies and/or oligonucleotide probes.

21. The method of claim 1, further comprising, prior to the administering:
measuring the concentration of the first biomarker in the sinonasal sample;
measuring the concentration of the second biomarker in the sinonasal sample;
comparing the concentration of the first biomarker to the reference diagnostic range threshold for the first biomarkers; and
comparing the concentration of the second biomarker to the reference diagnostic range threshold for the second biomarker.
